(19) United States
(12) Patent Application Publication
(10) Pub. No.: US 2025/0264460 A1
(43) Pub. Date: Aug. 21, 2025

(54) METHOD OF MEASURING LEVEL OF IMMUNE SUPPRESSION

(71) Applicant: THE JOHNS HOPKINS UNIVERSITY, Baltimore, MD (US)

(72) Inventors: Justin R. Bailey, Timonium, MD (US); Elizabeth Thompson, Phoenix, MD (US); Andrea Lynn Cox, Lutherville, MD (US); Katie Cascino, Baltimore, MD (US)

(21) Appl. No.: 18/855,981

(22) PCT Filed: Apr. 12, 2023
(86) PCT No.: PCT/US2023/065680

§ 371 Date: Oct. 10, 2024

Related U.S. Application Data
(60) Provisional application No. 63/330,127, filed on Apr. 12, 2022.

(51) Int. Cl. G01N 33/50

(52) U.S. Cl. CPC G01N 33/5091; G01N 2333/70535; G01N 2333/70553; G01N 2333/70596; G01N 2333/91057; G01N 2333/91215; G01N 2800/52

(57) ABSTRACT

Provided herein are methods of measuring effects of immune suppression in a subject, the method comprising (a) quantifying a number of immune cells in a biological sample of the subject: (b) determining an expression level of a protein from the biological sample; and (c) analyzing the number of immune cells and the expression level of the protein, thereby measuring the effects of immune suppression in the subject.

### CROSS-REFERENCE TO RELATED APPLICATIONS

This application claims priority to U.S. Provisional Patent Application No. 63/330,127, filed on Apr. 12, 2022, which is incorporated herein by reference in its entirety.

### FEDERALLY SPONSORED RESEARCH OR DEVELOPMENT

This invention was made with government support under grants CA260492 and AI138897 awarded by the National Institutes of Health. The government has certain rights in the invention.

### TECHNICAL FIELD

Described herein are methods for measuring effects of immune suppression by assessing combinations of cell phenotypes and metabolic function.

### BACKGROUND

Vaccines against SARS-COV-2 dramatically reduce COVID-19 severity at the population level, but not all individuals benefit equally. Immunocompromised individuals have higher COVID-19 mortality rates, diminished antibody titers, and higher rates of breakthrough infections following vaccination, with the lowest seroconversion rates occurring in solid organ transplant recipients (SOTRs). The benefits of a third vaccine dose in SOTRs with negative or low anti-spike(S) IgG titers after two vaccine doses has been previously demonstrated. However, even following a third dose of vaccine, a subset of SOTRs fail to develop SARS-COV-2 spike specific antibodies at titers thought to be protective. To gain additional insight into factors associated with vaccine response or nonresponse in immunocompromised people, clinical parameters and characterized global and antigen-specific B cell responses following second and third dose COVID-19 vaccination in SOTRs were assessed utilizing a high dimensional flow cytometry panel designed to evaluate immunologic and metabolic phenotypes at the single-cell level. Due to limited cross-reactivity with seasonal coronaviruses, the novel SARS-COV-2 vaccine provides a unique opportunity to evaluate naïve B cell priming in immunosuppressed people. Moreover, little is known about the metabolic landscape of successful or impaired human B cell responses. Using this approach, it was found that SOTRs utilize lipid-oxidizing alternative lineage CD11c+B cells, a minor population in healthy controls (HCs), as the primary pathway to successful vaccine responses and that high dose mycophenolate mofetil (MMF) administration inhibits this alternative pathway. These data highlight a novel pathway to vaccine responsiveness in some SOTRs and a mechanism for vaccine failure in SOTRs receiving high dose MMF.

### SUMMARY

Provided herein are methods of measuring effects of immune suppression in a subject, the method comprising (a) quantifying a number of immune cells in a biological sample of the subject; (b) determining an expression level of a protein from the biological sample; and (c) analyzing the number of immune cells and the expression level of the protein, thereby measuring the effects of immune suppression in the subject.

In some embodiments, the subject is immunocompromised. In some embodiments, the subject is a solid organ transplant recipient (SOTR). In some embodiments, the subject has an autoimmune disease. In some embodiments, the subject is administered an immunosuppressive agent. In some embodiments, the immunosuppressive agent is mycophenolate motefil (MMF).

In some embodiments, the immune cell is a B cell. In some embodiments, the B cell is a CD11c+B cell.

In some embodiments, the protein is an immunometabolic marker on a B cell. In some embodiments, the immunometabolic marker is selected from the group consisting of CD19, CD20, CD10, CD27, CD21, IgM, IgD, CD24, CD38, CD43, CD86, CXCR5, CD11c, CD39, FcRL5, BTLA, CD22, CD32, CD3, CD14, CPT1a, Hexokinase II, VDAC1, Tomm20, GLUT1, or any combinations thereof. In some embodiments, the immunometabolic marker is selected from CPT1a, HK2, CD11c, FcRL5, CD39, or any combinations thereof. In some embodiments, the immunometabolic marker is CPT1a.

In some embodiments, the analyzing step (c) comprises identifying the number of immune cells and the expression level of the protein, and using the number of immune cells and the expression level of the protein in combination to determine an immune response in the subject. In some embodiments, the immune response comprises determining the subject's ability to respond to a vaccination or infection. In some embodiments, the vaccination is a COVID-19 vaccination.

In some embodiments, the analyzing step (c) comprises performing flow cytometry. In some embodiments, the biological sample is blood plasma.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1K show reduced class-switched S-specific titers and B cells in solid organ transplant recipients (SOTRs) following two vaccine doses. Anti-S titers and S-specific B cells were evaluated in mRNA vaccinated healthy controls (HCs) (n=10) at peak response two weeks post dose two (FIG. 1A), 6 months post dose two (FIGS. 1B-1K), and SOTRs (n=44) 3-4 months post dose two, prior to dose three. FIG. 1A shows total anti-S IgG titers. Positive anti-S titers as determined by manufacture's cut off is denoted by dotted line. FIG. 1B shows anti-S IgG subclasses and IgA titers. FIG. 1C shows anti-S IgM titers. FIG. 1D shows representative staining of S-specific B cells. FIGS. 1E-1F show frequency of class-switched S-specific B cells as frequency of total class-switched B cells (FIG. 1E) or total PBMCs (FIG. 1F). Detectable threshold denoted by dotted line. FIG. 1G shows correlation between anti-S IgG titers and frequency of S-specific B cells out of total class-switched B cells. FIGS. 1H-1I show frequency of unswitched S-specific B cells as frequency of total unswitched B cells (FIG. 1H) or total PBMCs (FIG. 1I). FIG. 1J shows ratio of S-specific B cells with switched to unswitched phenotype. FIG. 1K shows proportion of participants with S-specific B cells or anti-S IgG titers above positive threshold.

FIGS. 2A-2J show that a third vaccine dose significantly increases anti-S titers and S-specific B cell frequencies in SOTRs. S-specific titers and anti-S B cells were evaluated in SOTRs (n=44) 3-4 months post dose two (Pre) and two weeks post dose three (Post). In all panels, responders (positive IgG titers after dose 3) and non-responders are indicated. FIG. 2A shows change in total anti-S IgG titers. FIG. 2B shows change in anti-S IgG subclasses, IgA, and IgM titers. FIG. 2C shows representative staining of S-specific B cells and frequency of class-switched or unswitched S-specific B cells. FIG. 2D shows anti-S IgG titers in non-responders (NR), responders (R) or healthy controls (HCs) pre-dose 3. FIG. 2E shows anti-S IgG titers in NR, R, or HCs at peak response post dose three (SOTRs) or dose two (HCs). FIGS. 2F-2H show correlation between anti-S IgG titers and frequency of S-specific B cells as indicated. FIGS. 21-2J show anti-S IgG titers following dose three stratified by immunosuppression received.

FIGS. 3A-3J show CD11c+B cells dominate response to COVID vaccination in SOTRs. FIG. 3A shows concatenated flow cytometry data depicted as UMAP projection of CD19+B cells colored by cluster annotation as determined by FlowSOM using a subset of the cohort (n=10 HCs, 4 NRs, 17 Rs). FIG. 3B shows frequency of FlowSOM clusters segregated by response. FIG. 3C shows a heatmap depicting mean scaled expression of all markers in flow cytometry panel and annotated according to FlowSOM cluster. FIG. 3D shows standard gating strategy to identify CD11c-B cells in complete cohort (n=10 HCs, 12 NRs, 32 Rs) and overlay of S-specific B cells from SOTRs and HCs. FIG. 3E shows frequency of CD11c+B cells out of total B cells. FIG. 3F shows frequency of CD11c+B cells out of S-specific B cells. FIG. 3G shows correlation of CD11c- and CD11c-S-specific B cells post-dose three with anti-S IgG titers post-dose three. FIG. 3H shows representative plot of CPT1a expression in CD11c+ and CD11c-B cells. FIGS. 31-3.J show mean fluorescent intensity (MFI) of CPT1a on CD11c-, CD11c+ (FIG. 3I left and right panel, respectively) or total class-switched (FIG. 3J) B cells.

FIGS. 4A-4J show high dose MMF inhibits mitochondrial fatty acid oxidation. FIGS. 4A-4B show normalized mean fluorescent intensity (MFI) of CD11c and CPT1a on total class-switched B cells segregated by dose of MMF; high (>1000), low (<1000), or none. FIG. 4C shows oil red O staining of lipid droplets in PBMCs stained directly post thaw from a SOTR not on MMF or on high dose MMF. FIGS. 4D-4E show oxygen consumption rate (OCR) following mitochondrial stress test of PBMCs plated directly post thaw from SOTRs or HCs (FIG. 4D) or healthy PBMCs treated in vitro with high or low dose MPA 48 hours prior to plating (FIG. 4E).

FIG. 4F shows oil red O staining of healthy PBMCs treated with high dose MPA (5 μM) or untreated FIG. 4G shows OCR of healthy PBMCs treated with high or low dose MPA 48 hours to plating and exposed to etomoxir (2 μM) 1 hour prior to measurement. FIG. 4H shows OCR of healthy PBMCs treated with high dose MPA 48 hours prior to plating then exposed to Palmitate (165 μM) 20 minutes prior to measurement. FIG. 4I shows fold change in OCR when healthy PBMCs were supplied with indicated substrates. FIG. 4J shows overview of proposed mechanism.

FIGS. 5A-5E show clinical features associated with responders (R) and non-responders (NR). FIG. 5A shows time since transplant (years). FIG. 4B shows age of participant (years). FIG. 5C shows anti-S IgG titers after dose three segregated by three dose vaccine regimens. Moderna (M), Pfizer (P), Johnson and Johnson (J). FIG. 5D shows vaccine regimen segregated by responders and non-responders. FIG. 5E shows anti-S IgG titers after dose three segregated by transplanted organ. Data presented as individual patients and mean.

FIGS. 6A-6D show that traditional B cell gating strategy does not predict response. FIG. 6A shows representative gating tree to identify the following B cell subsets: Transitional, Mature, Non-plasmablasts, Plasmablasts, Unswitched, Class-Switched, Activated Memory (AM), Intermediate Memory (IM), Atypical Memory (AtM), Resting Memory (RM), IgM only Memory, IgD only Memory, Naïve, and Marginal Zone (MZ). FIGS. 6B-6C show frequency of defined subsets out of total peripheral blood mononuclear cells (PBMCs) in healthy controls (HCs), responders, and non-responders pre-dose 3 (FIG. 6B) and post-dose 3 (FIG. 6C). FIG. 6D shows frequency of indicated B cell subset out of total B cells.

FIGS. 7A-7F show multivariate analysis of B cell phenotype. FIGS. 7A-7C show normalized mean fluorescent intensity (MFI) of all evaluated proteins on bulk B cells (FIG. 7A), unswitched B cells (FIG. 7B), and class-switched B cells (FIG. 7C) together with the frequency of class-switched S-specific B cell, unswitched S-specific B cells, and anti-S IgG titers two weeks post dose three were analyzed using a multivariate nonparametric Spearman's test and 2-tailed P value. Correlations with a nonsignificant P value (>0.05) had Spearman's coefficient changed to 0, and remaining values underwent 2-way hierarchical clustering. FIGS. 7D-7F show normalized MFI of proteins that significantly correlated with development of anti-S IgG titers or S-specific B cells in bulk B cells (FIG. 7D), Unswitched B cells (FIG. 7E), or Class-switched B cells (FIG. 7F), stratified by response and time point. Non-responders (NR), responders (R) were evaluated pre-dose three (pre) or two weeks post-dose three (post) and healthy controls (HC) were evaluated 6 months post dose two.

FIGS. 8A-81 show high dose MPA induces mitochondrial unresponsiveness. FIG. 8A shows Cell Trace Violet staining of T cells stimulated with anti-CD3/anti-CD28 or B cells stimulated with CD40Ligand, CpG, and anti-IgG/IgM/IgA for 6 days in the presence of MPA at indicated dose. FIG. 8B shows summary of T cell and B cell proliferation (n=3). FIG. 8C shows viability of PBMCs 48 hours after treatment with 5 μM MPA. FIGS. 8D-8G show healthy PBMCs were treated with 5 μM MPA for 24 hours in complete media then rested overnight in low-nutrient media prior to analysis. Oxygen consumption rate (OCR) of exposed to long chain fatty acid palmitate (165 μM) (FIG. 8D), medium chain fatty acid octanoic acid (200 μM) (FIG. 8E), glucose (10 mM) and pyruvate (1 mM) (FIG. 8F), or glutamine (2 mM) (FIG. 8G) 20 minutes prior to analysis. FIGS. 8H-81 show fold change in baseline (FIG. 8H) or maximal (FIG. 81) OCR when healthy PBMCs were supplied with indicated substrates.

FIG. 9 shows an exemplary proposed mechanism of response to vaccination in SOTRs. Solid organ transplant recipients receiving immunosuppressive agents utilize alternative lineage B cells as a salvage pathway to generate anti-S antibodies. These cells highly express CPT1a and are supported by increased fatty acid oxidation induced by immunosuppression, including low dose MMF. High dose MMF inhibits mitochondrial function and restricts alternative lineage B cell expansion, leading to a lack of anti-S IgG titers.

FIG. 10 shows an exemplary schematic of a proposed mechanism of response to vaccination in SOTRs.

FIGS. 11A-11C show 3^{rd} COVID-19 vaccine dose increases class-switched and anti-S titers and S-specific B cell frequencies. FIG. 11A shows that following the 3^{rd} vaccine dose, anti-S IgG titers increased significantly with 73% of SOTRs considered responders. FIG. 11B shows representative staining and enumeration of S-specific B cells demonstrates increase in class-switched and unswitched compartment following 3^{rd} dose. FIG. 11C shows all participants who failed to respond to a 3^{rd} dose received mycophenolate mofetil (MMF) immunosuppressive drug.

FIGS. 12A-12G show CPT1a+CD11c+B cells support response to COVID-19 vaccination following transplantation. FIG. 12A shows FlowSOM clustering identified a cluster of B cells (CD11c+CPT1a+) present in responders prior to dose 3 that expanded after dose 3. FIG. 12B shows representative gating for identified cluster of B cells. FIG. 12C shows total CD11c+B cells are increased in SOTR responders (R) compared to HC and non-responders (NR). FIG. 12D shows while ˜80% of the S-specific B cells in SOTRs were CD11c+, only 10-20% of S-specific B cells were CD11c+ in HCs. FIG. 12E shows both CD11c+S-specific B cells and CD11c-S-specific B cell frequencies correlated with anti-IgG titers in SOTRs. FIG. 12F shows CD11c-cells isolated from SOTR R produced higher levels of anti-S IgG following polyclonal stimulation for four days. FIG. 12G shows CD11c+ cells and CD11c-cells isolated from SOTR NR.

FIGS. 13A-13B show scRNA seq reveals increased Oxidative Phosphorylation and Fatty Acid Oxidation in CD11c-B cells from SOTRs. FIG. 13A shows gene set enrichment was performed on total B cells comparing CD11c+B cells to CD11c-B cells from SOTRs and HCs. FIG. 13B shows gene set enrichment was performed on CD11c+B cells comparing SOTR to HCs.

FIGS. 14A-14E show immunosuppression induces lipid synthesis and accumulation. FIG. 14A shows genes associated lipid synthesis are increased in total B cells from SOTRs (R or NR) in scRNA seq. FIG. 14B shows PCA plot of lipidomics performed on total B cells derived from SOTRs or HCs. FIG. 14C shows hierarchical clustering of top 20 differentially expressed lipids in B cells from SOTRs and HCs demonstrate clear segregation in lipid profiles. FIG. 14D shows accumulation of lipid droplets as measured by Oil Red O in PBMCs from HC or SOTR. There are higher levels of Oil Red O staining in individuals on high dose MMF. FIG. 14E shows accumulation of lipid droplets is seen by Oil Red O staining in healthy cells treated with high dose of mycophenolic acid (active ingredient in MMF).

FIGS. 15A-15H show immunosuppression induces basal mitochondrial lipid oxidation that is inhibited by high dose MMF. FIG. 15A shows PBMCs from SOTRs receiving low dose MMF demonstrated higher basal respiration compared to HCs and PBMCs from patients on high dose MMF exhibited reduced oxygen consumption rate (OCR). FIGS. 15B-15F show healthy PBMCs were treated with high (5 μM) and low dose (0.5 μM) MPA in vitro. FIG. 15B shows results consistent with ex vivo results, low dose MPA directly induced increased OCR. FIGS. 15C-15D show addition of etomoxir completely reversed the increase in OCR. FIG. 15E shows following treatment with high does MPA PBMCs are not able to oxidize exogenous long-chain fatty acid palmitate. FIG. 15F shows high dose MPA inhibits mitochondrial oxidation of various substrates. FIGS. 15G-15H show proposed mechanism of high vs low dose MPA on mitochondrial metabolism.

FIGS. 16A-16C show low dose MPA induces CPT1a-dependent FAO in B cells but not T cells. FIG. 16A shows T cells express significantly lower levels of CPT1a than CD11c- and CD11c-B cells. FIG. 16B shows only B cells increased their OCR in response to low dose MPA, and high dose MPA blunted the increased OCR in B cells. FIG. 16C shows inhibiting CPT1a with etomoxir reduced OCR to baseline levels in B cells but had no effect on T cells. These data demonstrate that high-level CPT1a expression is required for enhanced FAO following low dose MPA treatment and CD11c+B cells that express the highest levels of CPT1a are best equipped to support the unique metabolic flux.

FIGS. 17A-17E show IFNg supports CD11c+B cell differentiation and response to vaccination. FIG. 17A shows gene set enrichment on CD11c+B cells from SOTR responders compared to non-responders demonstrates increased IFNg signaling in responders. FIG. 17B shows plasma IFNg levels are increased in responders. FIG. 17C shows S-specific T cells that produce IFNg are increased in responders. FIG. 17D shows healthy B cells were provided polyclonal stimulation (CpG, IL-21, CD40L) for four days in the presence or absence of low dose MPA and IFNg. The frequency of CD11c+B cells in culture was evaluated at day 4. IFNg and MPA combined induced the highest levels of CD11c+B cells. FIG. 17E shows proposed dichotomy of lipid synthesis driving CD11c expression on B cells and requirement for IFNg to generate detectable antibody responses in SOTRs.

### DETAILED DESCRIPTION

The COVID-19 pandemic highlights the need to understand successful vaccine responses in immunocompromised individuals, including solid organ transplant recipients (SOTRs). Vaccines against SARS-COV-2 dramatically reduce COVID-19 severity at the population level, but not all individuals benefit equally. It has been shown that immunocompromised individuals have higher COVID-19 mortality rates, diminished antibody titers, and higher rates of breakthrough infections following vaccination, with the lowest seroconversion rates occurring in solid organ transplant recipients (SOTRs).

Immune suppression is required in treating patients who have undergone solid organ transplantation or who have autoimmune diseases. This immune suppression is induced by medications (singly or in combination) designed to suppress the immune response. A flow cytometry-based technology can be used to quantify the degree of immune cell suppression by simultaneously assessing combinations of cell phenotypes and metabolic function. It has been demonstrated that the combination of these parameters define levels of immune suppression in humans. A combination of cell surface molecules can be used to define the metabolic activity of a variety of immune cells that are correlated with immune system function, including the ability to respond to vaccination. For example, this technique identified in patients on immunosuppressive agents characteristics of B cells associated with maintained ability to respond to vaccination by generation of antibodies (e.g., CD11c positive B cells with high levels of carnitine palmitoyltransferase 1a (CPT1a), the rate limiting enzyme for fatty acid oxidation). In some embodiments, measurement of these cellular parameters, including the percentage of CD11c positive cells with high CPT1a, permits titration of immunosuppressive agents to a level that suppresses sufficiently to prevent organ rejection or autoimmune disease while maintaining enough immune cell function to respond to vaccination or infection.

Provided herein are methods of measuring effects of immune suppression in a subject, the method including (a) quantifying a number of immune cells in a biological sample of the subject; (b) determining an expression level of a protein from the biological sample; and (c) analyzing the number of immune cells and the expression level of the protein, thereby measuring the effects of immune suppression in the subject. In some instances, the methods used herein include flow cytometry. In some instances, determining an expression level of a protein can include chemiluminescence or fluorescence techniques. In some embodiments, determining an expression level of a protein can include immunological-based methods (e.g., quantitative enzyme-linked immunosorbent assays (ELISA), Western blotting, or dot blotting) wherein antibodies are used to react specifically with entire proteins or specific epitopes of a protein. In some embodiments, determining an expression level of a protein can include immunoprecipitation of the protein.

Various non-limiting aspects of these methods are described herein, and can be used in any combination without limitation. Additional aspects of various components of methods for preventing hearing loss in a subject, protecting the inner ear in a subject, or preventing cochlear hair cell death in a subject are known in the art.

It must be noted that, as used in the specification and the appended claims, the singular forms “a,” “an,” and “the” include plural referents unless the context clearly dictates otherwise.

As used herein, the term “biological sample” refers to a sample obtained from a subject for analysis using any of a variety of techniques including, but not limited to, biopsy, surgery, and laser capture microscopy (LCM), and generally includes cells and/or other biological material from the subject A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer.

Biological samples can also include immune cells. Sequence analysis of the immune repertoire of such cells, including genomic, proteomic, and cell surface features, can provide a wealth of information to facilitate an understanding the status and function of the immune system. Examples of immune cells in a biological sample include, but are not limited to, B cells, T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hypersegmented neutrophils), monocytes/macrophages, mast cells, thrombocytes/megakaryocytes, and dendritic cells.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

As used herein, the term “subject” refers to an organism, typically a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

### Immune Suppression

Immunosuppression is a reduction of the activation or efficacy of the immune system. Some portions of the immune system itself have immunosuppressive effects on other parts of the immune system, while immunosuppression may occur as an adverse reaction to treatment of other conditions. In some embodiments, immune suppression can be deliberately induced to prevent the body from rejecting an organ transplant. Additionally, it can be used for treating graft-versus-host disease after a bone marrow transplant, or for the treatment of auto-immune diseases such as systemic lupus erythematosus, rheumatoid arthritis, Sjögren's syndrome, or Crohn's disease. In some embodiments, immune suppression can be done by using medications, but may also involve surgery (splenectomy), plasmapheresis, or radiation. In some embodiments, immune suppression can be non-deliberate immunosuppression. For example, non-deliberate immunosuppression can occur in ataxia-telangiectasia, complement deficiencies, many types of cancer, and certain chronic infections such as human immunodeficiency virus (HIV). The unwanted effect in non-deliberate immunosuppression can be immunodeficiency that results in increased susceptibility to pathogens, such as bacteria and viruses. Immunodeficiency can also be a potential adverse effect of many immunosuppressant drugs. In some embodiments, immunosuppression can refer to both beneficial and potential adverse effects of decreasing the function of the immune system.

Provided herein are methods of measuring effects of immune suppression in a subject, the method comprising (a) quantifying a number of immune cells in a biological sample of the subject; (b) determining an expression level of a protein from the biological sample; and (c) analyzing the number of immune cells and the expression level of the protein, thereby measuring the effects of immune suppression in the subject. In some embodiments, the subject is immunocompromised. In some embodiments, the subject is solid organ transplant recipient (SOTR). In some embodiments, the subject has an autoimmune disease. In some embodiments, the subject is administered an immunosuppressive agent.

As used herein, an “immunocompromised subject” can refer to a person who is undergoing immunosuppression, or whose immune system is weak for other reasons (e.g., chemotherapy or HIV). In some embodiments, an immunocompromised subject can be a solid organ transplant recipient, wherein solid organ transplant means live-donor kidney transplants and transplants of the following organs from cadaveric donors: kidney, pancreas, liver, intestines, heart and lung. Solid organ transplant does not include the transplantation of stem cells, bone marrow, peripheral blood or cord blood.

In some embodiments, the subject has an autoimmune disease, wherein the autoimmune disease is a disease in which the body's immune system attacks healthy cells. For example, an autoimmune disease can be, but is not limited to, Type 1 diabetes, Rheumatoid arthritis (RA), Psoriasis/psoriatic arthritis, Multiple sclerosis (MS), Systemic lupus erythematosus (SLE), Inflammatory bowel disease, Addison's disease, Graves' disease, Sjögren's syndrome, Hashimoto's thyroiditis, Myasthenia gravis, Autoimmune vasculitis, Pernicious anemia, or Celiac disease.

As used herein, an “immunosuppressive agent” is a drug that suppresses the immune system and reduces the risk of rejection of foreign bodies such as transplant organs. Immunosuppressive agents can lower the immunity when there is increased risk of infection. In some embodiments, immunosuppressive agents are used as cancer chemotherapy, in autoimmune diseases such as rheumatoid arthritis, or to treat severe allergy. In some embodiments, an immunosuppressive agent can include, glucocorticoids, cytostatics, antibodies, or drugs acting on immunophilins. In some embodiments, an immunosuppressive agent can include a calcineurin inhibitor, an interleukin inhibitor, a TNF-aplha inhibitor, or other selective immunosuppressants. For example, an immunosuppressive agent can be, but is not limited to, prednisone, dexamethasone, hydrocortisone, nitrogen mustards (cyclophosphamide), nitrosoureas, platinum compounds, methotrexate, azathioprine and mercaptopurine, fluorouracil, dactinomycin, anthracyclines, mitomycin C, bleomycin, mithramycin, polyclonal antibodies, monoclonal antibodies, T-cell receptor directed antibodies, IL-2 receptor directed antibodies, ciclosporin, tacrolimus, sirolimus, everolimus, zotarolimus, interferons, opioids, TNF binding proteins, mycophenolate, or small biological agents. In some embodiments, the immunosuppressive agent is mycophenolate motefil (MMF).

As used herein, “immune cells” refer to cells of the immune system which can be categorized as lymphocytes (e.g., T cells, B cells, NK cells and NKT cells), neutrophils, and monocytes/macrophages. In some embodiments, an immune cell is a B cell, wherein a B cell (e.g., B-lymphocytes, CD19, or CD20 cells) is a specialized cell of the immune system whose major function is to produce antibodies (e.g., immunoglobulins or gamma-globulins). B-cells develop in the bone marrow from hematopoietic stem cells, and when mature, they can be found in the bone marrow, lymph nodes, spleen, some areas of the intestine, and the bloodstream. In some embodiments, the B cell is a transitional B cell, a Naïve B cell, a memory B cell, or a plasma B cell. In some embodiments, the B cell is a CD11c+B cell.

In some embodiments, an immune cell can express a protein (e.g., a cell surface molecule) that defines the metabolic activity of the immune cell that can be correlated with an immune system function (e.g., ability to respond to vaccination). In some embodiments, the protein is an immunometabolic marker on a B cell. In some embodiments, the immunometabolic marker is selected from the group consisting of CD19, CD20, CD10, CD27, CD21, IgM, IgD, CD24, CD38, CD43, CD86, CXCR5, CD11c, CD39, FcRL5, BTLA, CD22, CD32, CD3, CD14, CPT1a, Hexokinase II, VDAC1, Tomm20, GLUT1, or any combinations thereof. In some embodiments, the immunometabolic marker is selected from CPT1a, HK2, CD11c, FcRL5, CD39, or any combinations thereof. In some embodiments, the immunometabolic marker is CPT1a.

As used herein, an “immune response” can refer to a way the body defends itself against substances it sees as harmful or foreign. In an immune response, the immune system recognizes the antigens (e.g., foreign proteins) on the surface of substances or microorganisms (e.g., bacteria or viruses), and attacks and destroys, or tries to destroy, them.

In some embodiments, the analyzing step (c) includes identifying the number of immune cells and the expression level of the protein, and using the number of immune cells and the expression level of the protein in combination to determine an immune response in the subject. In some embodiments, the immune response comprises determining the subject's ability to respond to a vaccination or infection. In some embodiments, the vaccination is a COVID-19 vaccination. In some embodiments, the analyzing step (c) comprises performing flow cytometry. In some embodiments, the biological sample is blood plasma.

### EXAMPLES

### Example 1-Reduced Class-Switched S-Specific Titers and B Cells in SOTRs Following Two Vaccine Doses

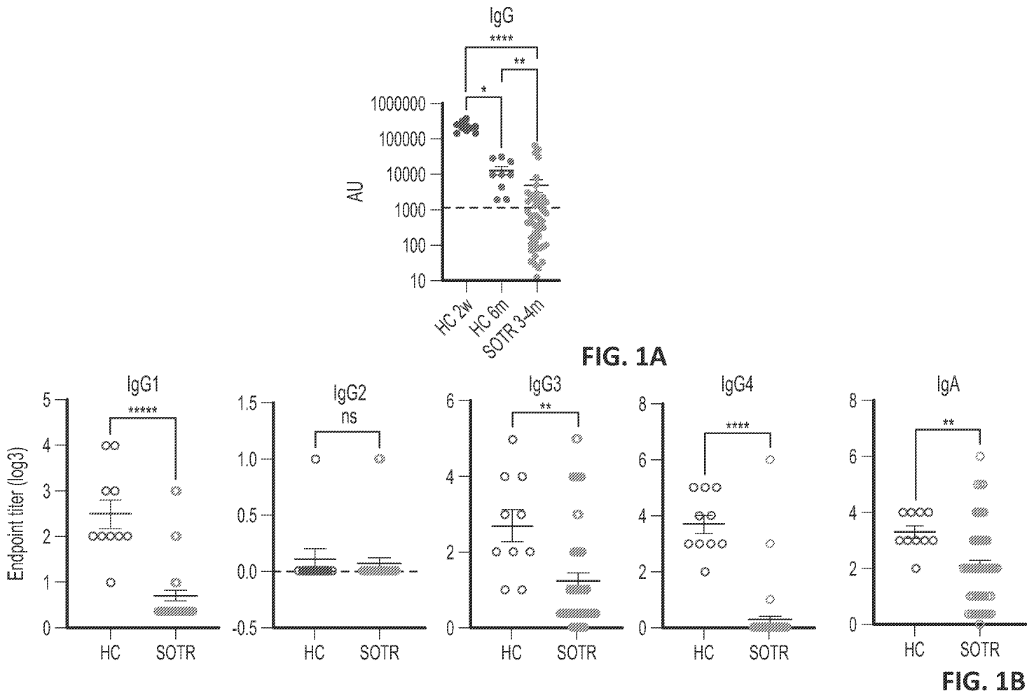

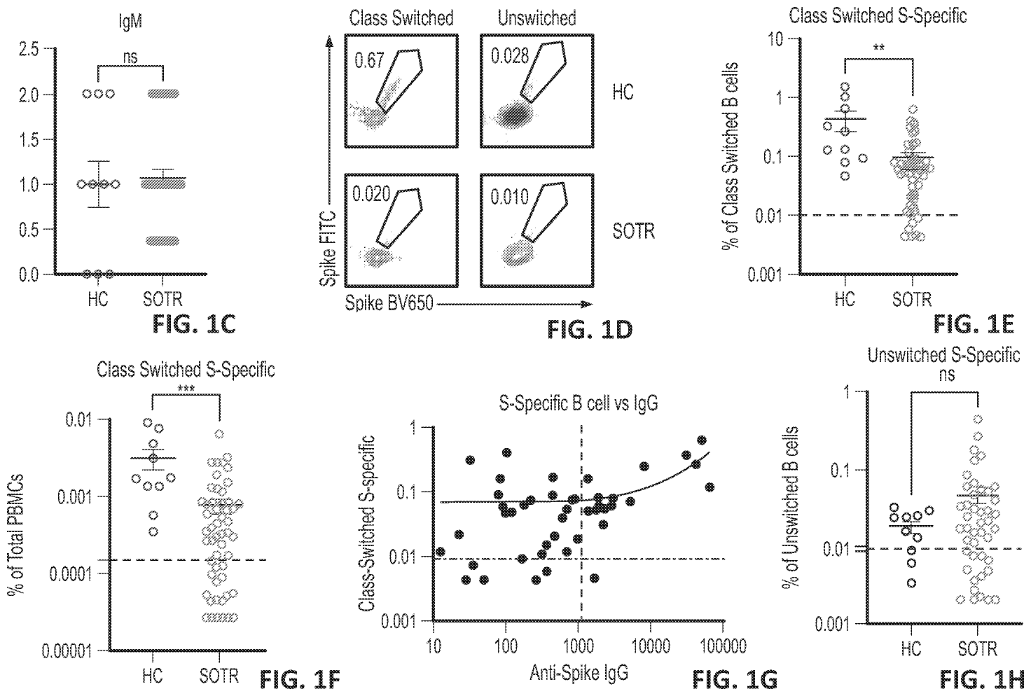

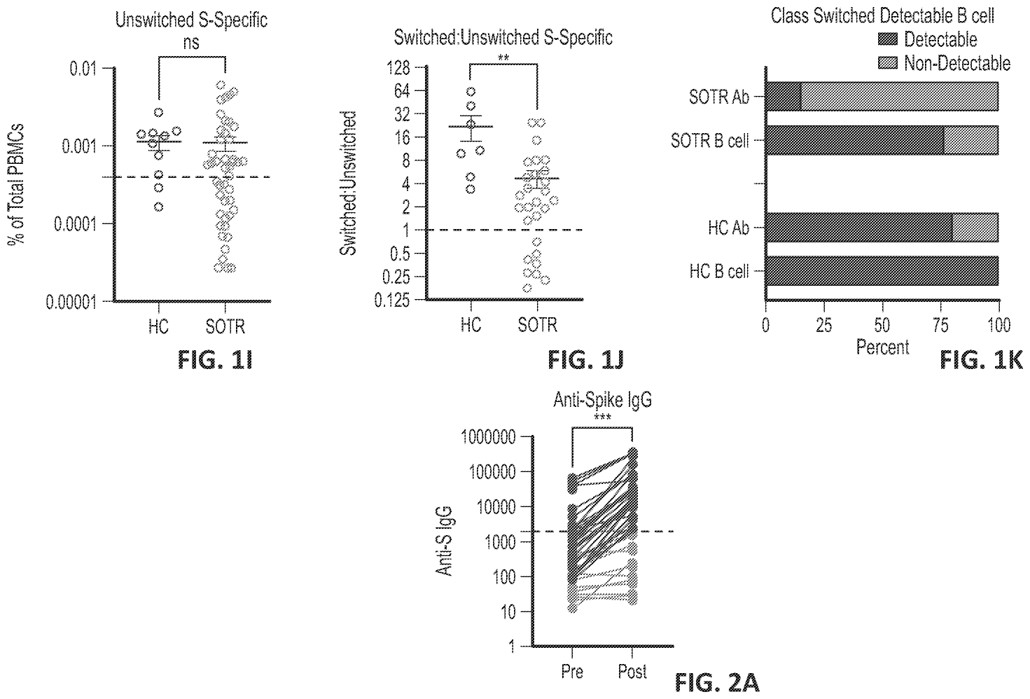

Peripheral blood from an observational cohort comprising 44 solid organ transplant recipients (SOTRs) with matched sample collection before and after third dose COVID-19 vaccination and 10 HCs following two doses of a COVID-19 vaccine was assayed for anti-S antibody titers and the development of S-specific memory B cells (Table 1). SOTRs demonstrated significantly lower anti-S IgG titers compared to HCs, with 85% of SOTRs having titers below the positive threshold following the standard two-dose regimen (FIG. 1A). Although HCs demonstrated a significant decline in anti-S IgG titers approximately 6 months following their second dose, all HCs maintained IgG titers above the positive threshold (FIG. 1A). Class-switched IgG subclass and IgA titers were also reduced in SOTRs relative to HCs (FIG. 1B). Developing B cells have variable dependence on T cell help, with class switching to IgG3 requiring less T cell help than other IgG subclasses. SOTRs showed less reduction in IgG3 than in IgG1 or IgG4 titers (FIG. 1B) and no difference from HCs in anti-S IgM titers (FIG. 1C), consistent with insufficient T cell help in persons on immunosuppressive therapy designed to inhibit T cell activation.

Following two doses, class switched S-specific B cells (CD19+IgM-IgD-) were detected at significantly lower levels in SOTRs relative to HCs (FIGS. 1D-1F), both as a percentage of total class switched B cells (FIG. 1E) and of total PBMCs (FIG. 1F). There was a significant correlation between the frequency of class switched S-specific B cells and anti-S IgG titers above the positive threshold (FIG. 1G). In contrast to class-switched B cells, there was no significant difference in the frequency of unswitched S-specific B cells (CD19+IgM+IgD+) between HCs and SOTRs (FIGS. 1H-1I). Consistent with these findings, the ratio of switched to unswitched S-specific B cells was significantly higher in HCs compared to SOTRs (FIG. 1J). Notably, although only 25% of SOTRs had a positive anti-IgG response following two vaccine doses, 76% had detectable class-switched S-specific B cells, indicating the possibility of boosting these B cells with a third dose (FIG. 1K). Together, these data demonstrate diminished class switching in SOTRs compared to HCs following the two-dose regimen, characterized by a failure to generate comparable class-switched S-specific B cells and Ab titers.

### Example 2-Third Vaccine Dose Significantly Increases Anti-S Titers and S-Specific B Cell Frequencies in SOTRs

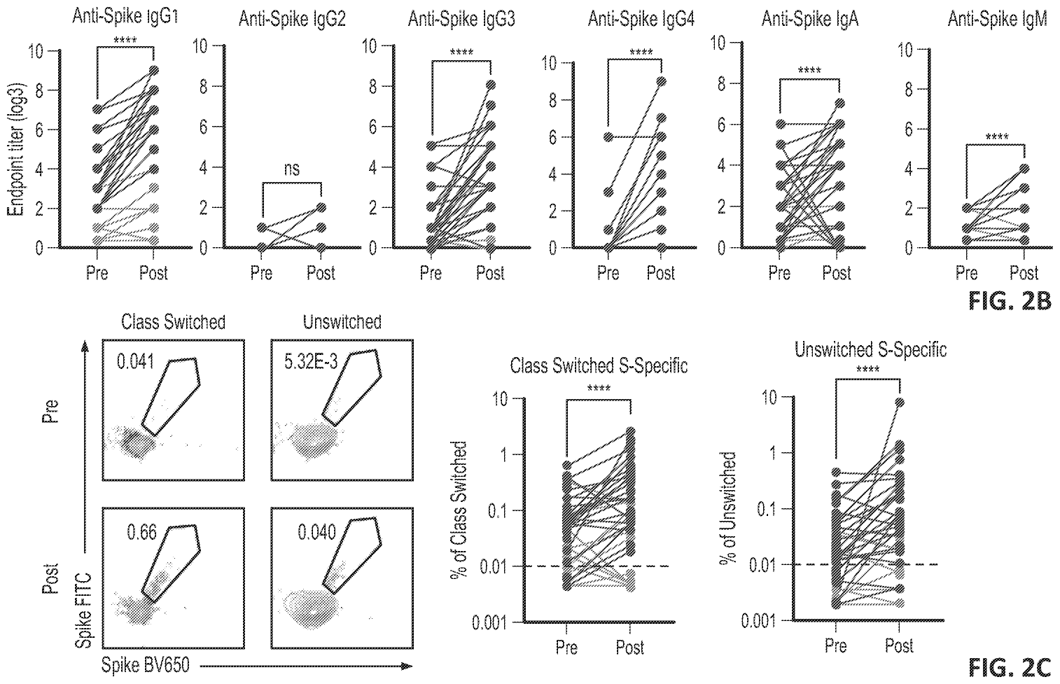

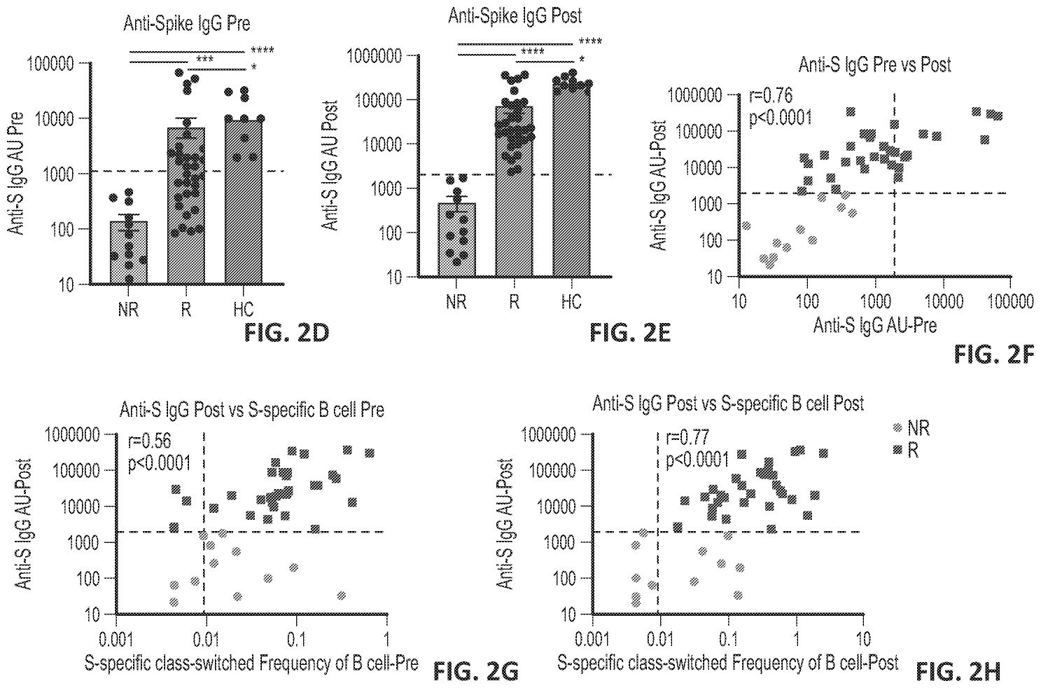

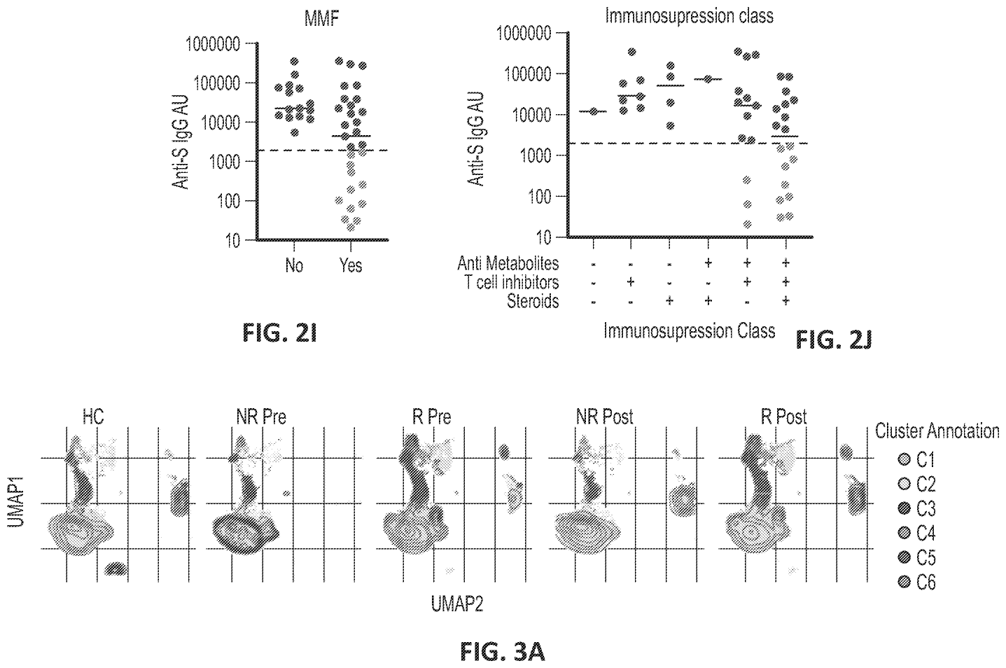

Following a third vaccine dose, anti-S IgG titers and S-specific B cell frequencies increased significantly, with 32 of 44 SOTRs (72.7%) considered responders (responders defined as individuals with antibody titers above the positive MSD manufacturer's threshold, FIGS. 2A-2C). SOTRs who responded to a third dose demonstrated significantly higher pre-dose anti-S IgG titers (FIG. 2D). Although increased, anti-S titers remained significantly lower than those of HCs at peak response (2 weeks post second dose) (FIG. 2E). Importantly, titers after dose 2 strongly correlated with the titers following dose 3 (FIG. 2F). The frequency of S-specific class-switched B cells pre (FIG. 2F) and post dose 3 (FIGS. 2G-2H) also correlated with anti-S IgG titers after dose 3.

TABLE 1
Participant Demographics and Clinical Characteristics,
Stratified by Spike IgG Response Two Weeks Post-D 3

| | Solid Organ Transplant Recipients | | | | | | | | |
|---|---|---|---|---|---|---|---|---|---|
| | | | Non- | | | | | | |
| | | | Detectable | | Detectable | | | Healthy | |
| | SOTRs | | Spike IgG | | Spike IgG | | | Controls | |
| | (n = 44) | | (n = 12) | | (n = 32) | | P-Value^{a} | (n = 10) | |
| Demographics | | | | | | | | | |
| Age, years, n (%) | | | | | | | 0.6 | | |
| 20-39 | 2 | (5) | 0 | (0) | 2 | (6) | | 2 | (20) |
| 40-59 | 17 | (39) | 6 | (50) | 11 | (34) | | 8 | (80) |
| 60+ | 25 | (57) | 6 | (50) | 19 | (59) | | 0 | (0) |
| Sex, n (%) | | | | | | | 0.3 | | |
| Male | 21 | (48) | 4 | (33) | 17 | (53) | | 7 | (70) |
| Female | 23 | (52) | 8 | (67) | 15 | (47) | | 3 | (30) |
| Race, n (%) | | | | | | | >0.99 | | |
| White | 42 | (95) | 12 | (100) | 30 | (94) | | — | |
| Asian | 1 | (2) | 0 | (0) | 1 | (3) | | — | |
| Black | 0 | (0) | 0 | (0) | 0 | (0) | | — | |
| Other | 1 | (2) | 0 | (0) | 1 | (3) | | — | |
| Transplant Characteristics | | | | | | | | | |
| Transplant Organ, n (%) | | | | | | | | | |
| Kidney | 25 | (57) | 8 | (67) | 17 | (53) | 0.03 | — | |
| Liver | 10 | (23) | 0 | (0) | 10 | (31) | | — | |
| Pancreas | 1 | (2) | 0 | (0) | 1 | (3) | | — | |
| Lung | 2 | (5) | 1 | (8) | 1 | (3) | | — | |
| Heart | 4 | (9) | 1 | (8) | 3 | (9) | | — | |
| Multi | 2 | (5) | 2 | (17) | 0 | (0) | | — | |
| Years Since Transplant, n (%) | | | | | | | 0.4 | | |
| <3 | 18 | (41) | 7 | (58) | 11 | (34) | | — | |
| 3-11 | 16 | (36) | 3 | (25) | 13 | (41) | | — | |
| 12+ | 10 | (23) | 2 | (17) | 8 | (25) | | — | |
| Immunosuppression | | | | | | | | | |
| Regimen, n (%)^{b} | | | | | | | | | |
| Corticosteroids | 23 | (52) | 9 | (75) | 14 | (44) | 0.09 | — | |
| Calcineurin Inhibitors | 37 | (84) | 9 | (75) | 28 | (88) | 0.4 | — | |
| mTOR Inhibitors | 7 | (16) | 2 | (17) | 5 | (16) | >0.99 | — | |
| Anti-Metabolites | 32 | (73) | 12 | (100) | 20 | (63) | 0.02 | — | |
| High Dose MMF or | 21 | (75) | 10 | (83) | 11 | (69) | 0.7 | | |
| MPA^{c} | | | | | | | | | |
| Triple | 14 | (32) | 6 | (50) | 8 | (25) | 0.2 | | |
| Immunosuppression^{d} | | | | | | | | | |
| Treated for Transplant Rejection | 1 | (2) | 1 | (8) | 0 | (0) | 0.3 | — | |
| Within Six Months Pre-D 1, n | | | | | | | | | |
| (%)^{e} | | | | | | | | | |
| Vaccine Information | | | | | | | | | |
| D 1 and D 2 Vaccine Type, n (%) | | | | | | | >0.99 | | |
| Pfizer/BioNTech | 22 | (50) | 6 | (50) | 16 | (50) | | 10 | (100) |
| Moderna | 22 | (50) | 6 | (50) | 16 | (5) | | 0 | (0) |
| Johnson and Johnson | 0 | (0) | | | | | | 0 | (0) |

| | | | | | | | | |
|---|---|---|---|---|---|---|---|---|
| D 3 Vaccine Type, n (%) | | | | | | | 0.2 | |
| Pfizer/BioNTech | 12 | (27) | 3 | (25) | 9 | (28) | | — |
| Moderna | 18 | (41) | 3 | (25) | 15 | (47) | | — |
| Johnson and Johnson | 14 | (32) | 6 | (50) | 8 | (25) | | — |
| Days between D 2 and D 3, median | 99 | (64-124) | 85 | (53-112) | 103 | (68-131) | 0.1 | — |
| (IQR) | | | | | | | | |

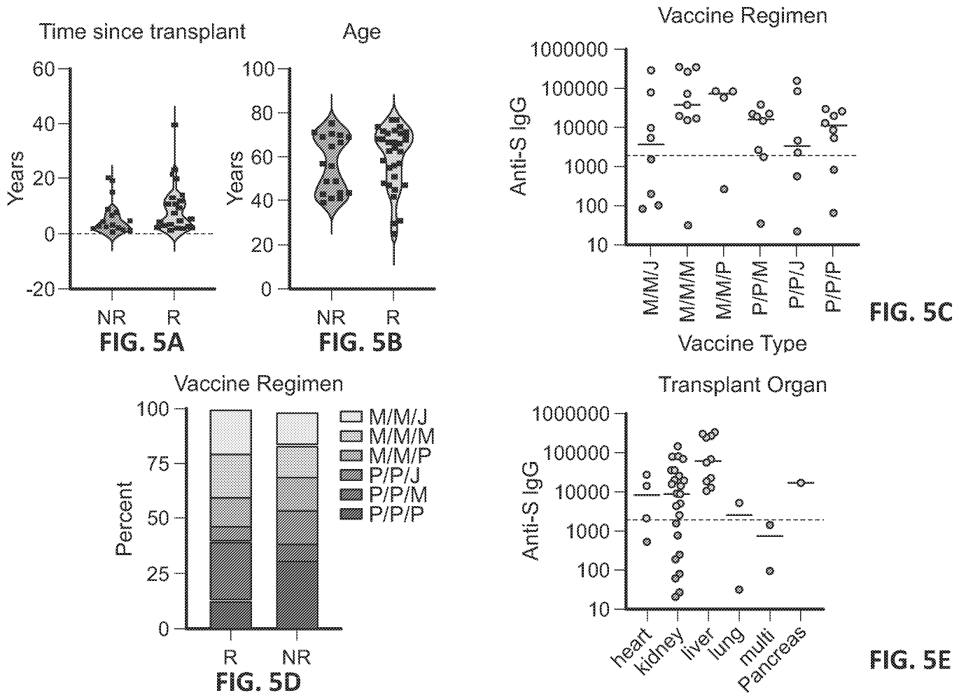

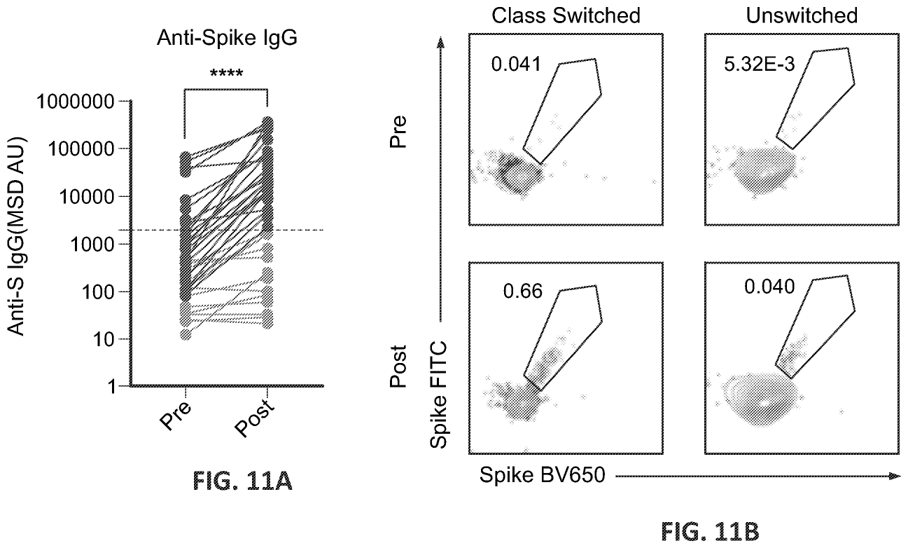

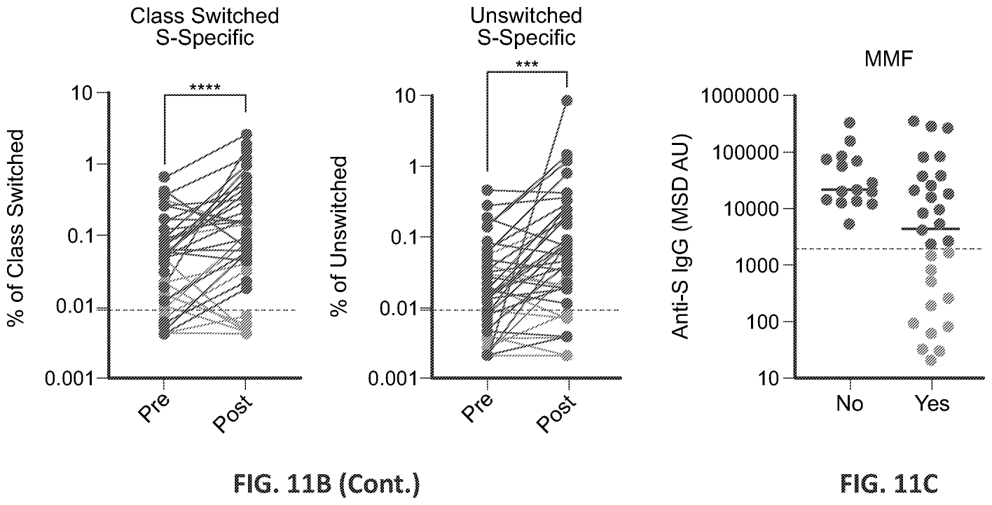

Clinical parameters differentiating responders from non-responders following the third dose were evaluated, as defined in FIG. 2A. There were no significant differences between responders and non-responders in the vaccine received, organ transplanted, age or time since transplant (FIGS. 5A-5E, Table 1). However, it was found that all participants who failed to respond to a third dose received a combination of mycophenolate mofetil (MMF) and a T cell activation inhibitor, including mTOR inhibitors and calcineurin inhibitors as described in Table 1 (FIGS. 21-2J). In contrast, participants on T cell activation inhibitors or steroids alone were able to respond. Treatment with MMF was specifically associated with a lack of response. Collectively, the data indicate that anti-S IgG titers or S-specific B cell frequencies following dose 2 strongly correlate with dose 3 responses, and that nonresponse is associated with MMF treatment (FIGS. 11A-11C).

### Example 3-Multivariate Analysis of B Cell Phenotype

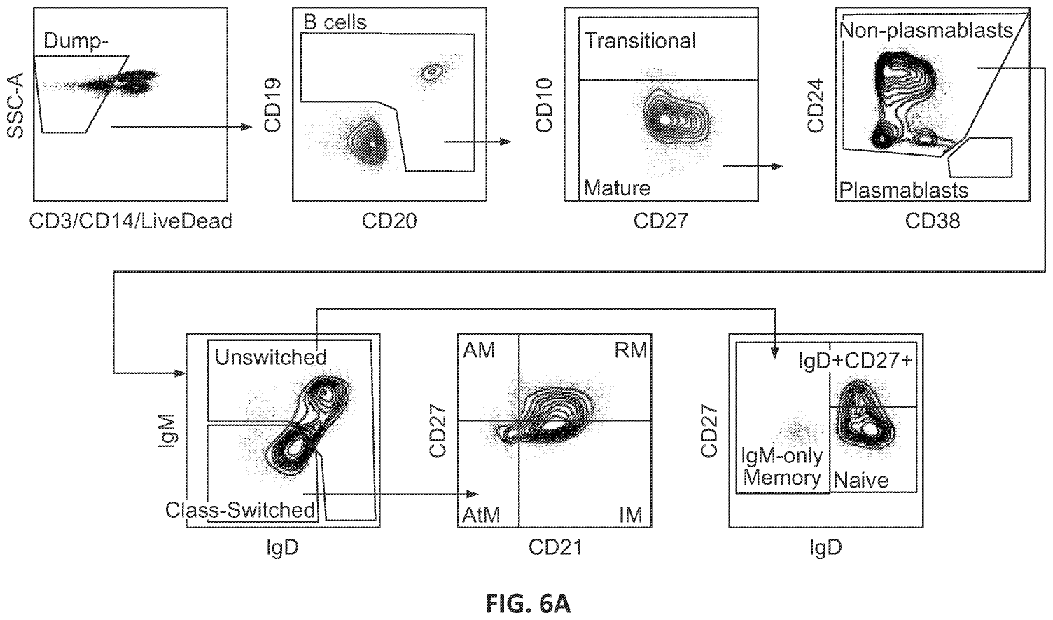

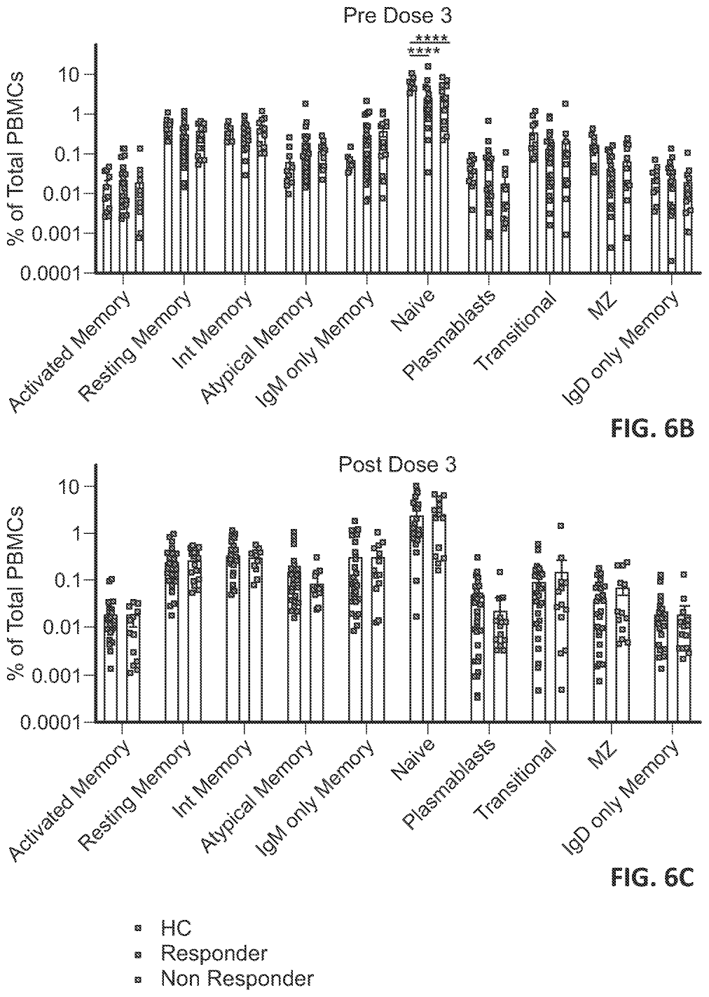

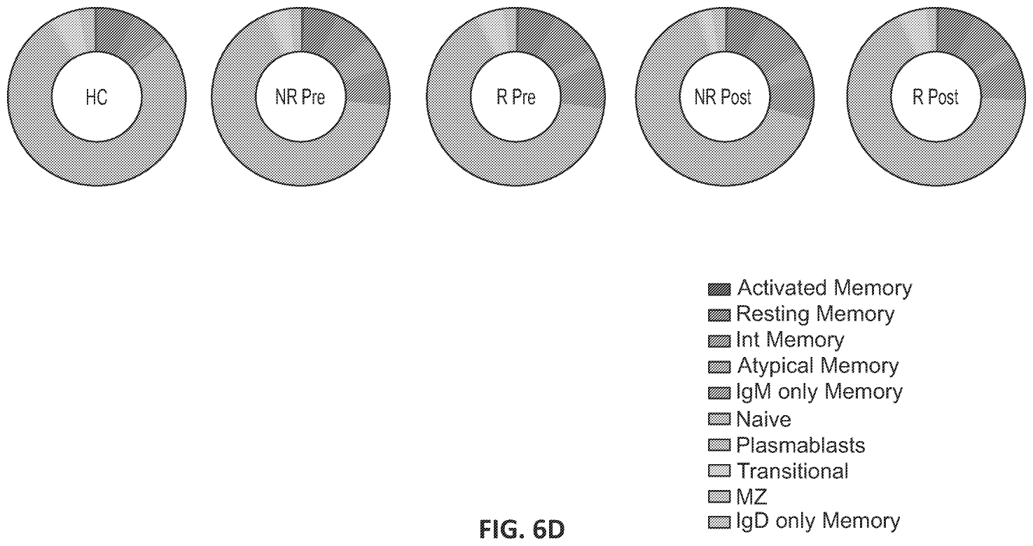

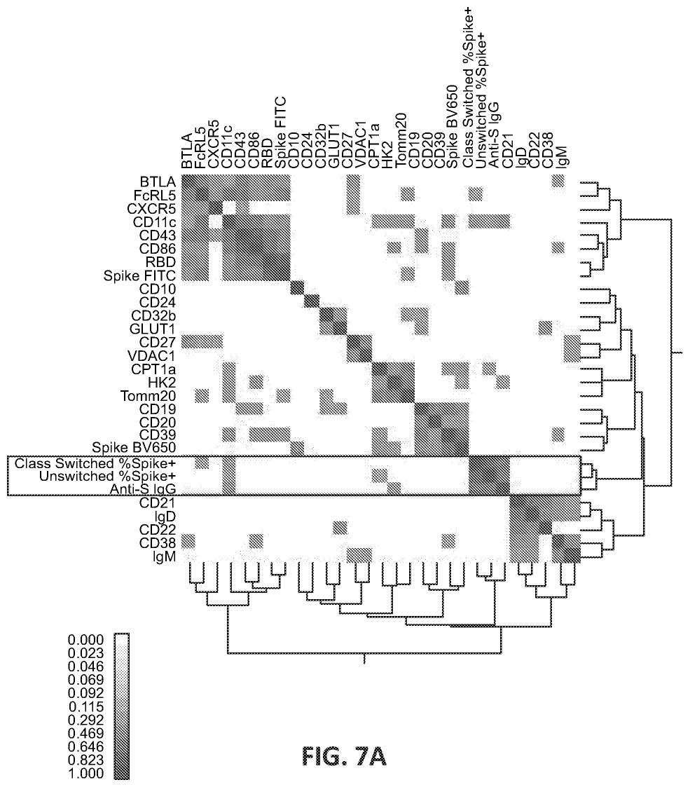

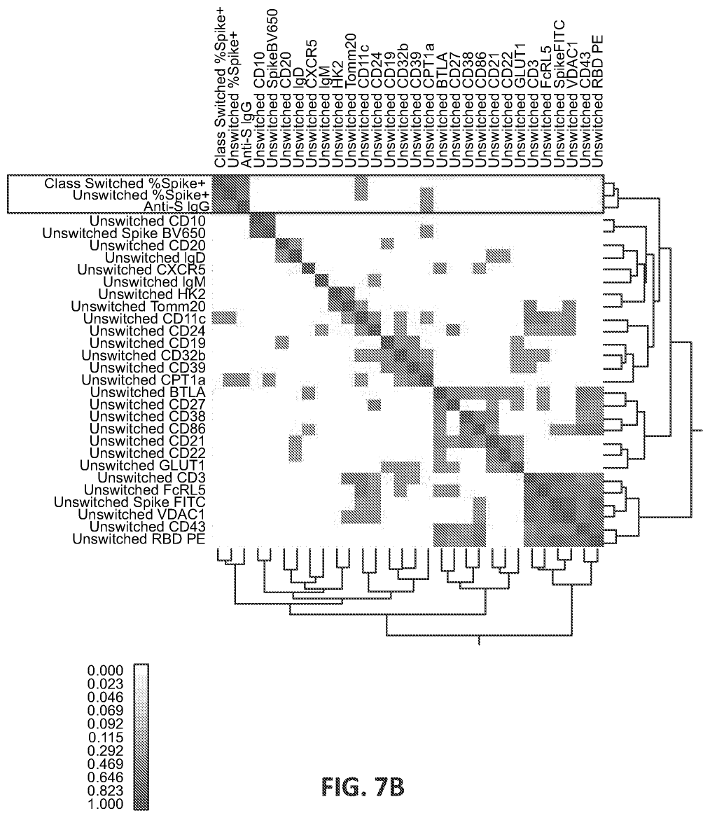

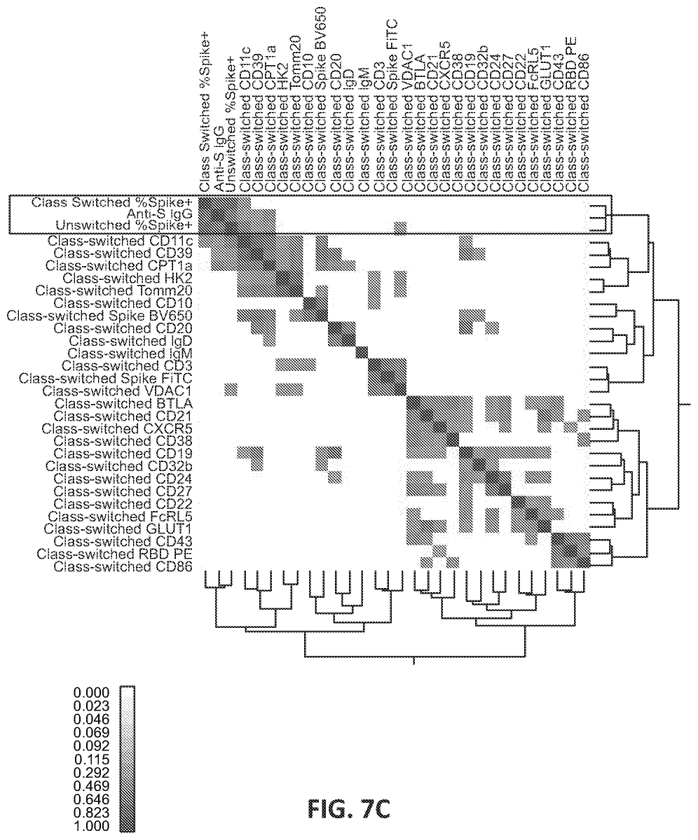

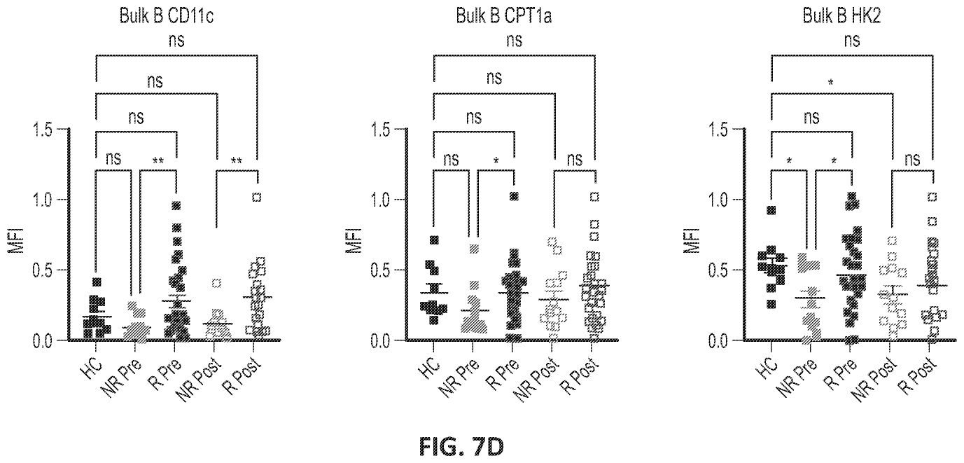

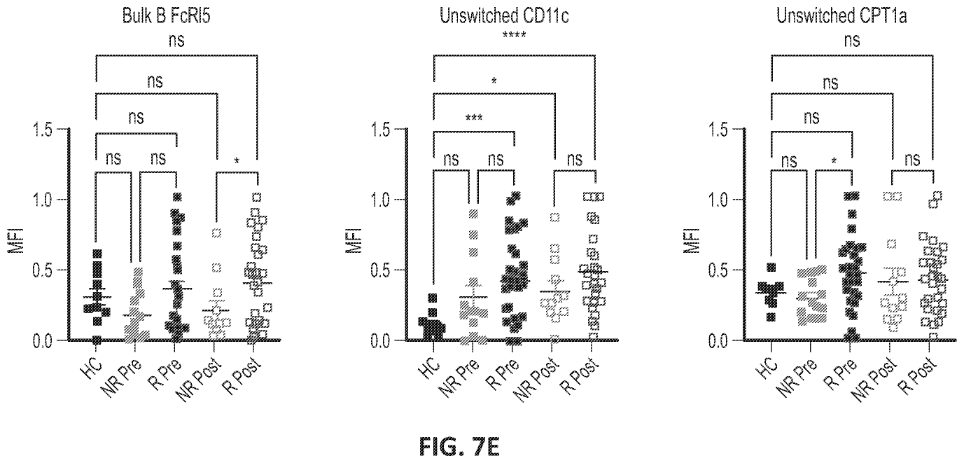

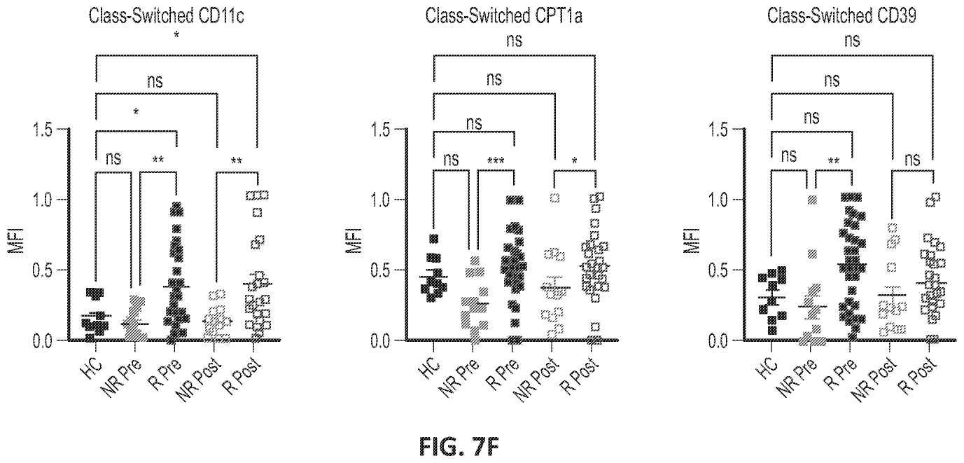

To understand the underlying mechanisms of successful vaccine responses in SOTRs, both biased and unbiased analyses on total B cells was performed by flow cytometry. Pathways and phenotypes that allowed responders to mount an immune response despite immunosuppression were identified, aiming to identify molecular targets for increasing anti-S titers in non-responders. Evaluating frequencies of traditional B cell subsets did not identify significant differences between responders and non-responders, before or after the third dose of vaccine (FIGS. 6A-6D). Multivariate correlative analysis was performed using protein expression of 26 immunometabolic markers on total B cells, unswitched B cells, or class-switched B cells prior to dose 3 with the development of anti-S IgG and S-specific B cells at the peak response post-dose 3 (FIGS. 7A-7C). In total, the expression of five proteins, carnitine palmitoyltransferase 1a (CPT1a), hexokinase-2 (HK2), CD11c, Fc receptor-like 5 (FcRI.5), and CD39, correlated with increased anti-S IgG and S-specific B cells post-dose 3 (FIGS. 7D-7F). The metabolic demands of antibody-producing B cells have historically been ill-defined. However, the unique utilization of fatty acid oxidation (FAO) in germinal center B cells have been highlighted to meet their high energetic and biosynthetic demands. CPT1a is the rate-limiting enzyme in FAO. Thus, its high expression in B cells of responding participants (FIG. 7F) suggests these cells are enabled to effectively oxidize fatty acids. Additionally, activated B cells have shown a dependence on glycolysis, potentially enhanced via increased HK2 expression. CD11c and FcRL5 expression has been associated with alternative lineage B cells, including atypical memory B cells. Atypical memory B cells have traditionally been identified in the context of chronic antigen stimulation. However, recent reports highlight that the CD11c+B cell compartment is more diverse than previously understood, comprising part of the response to vaccination in healthy individuals. Finally, the ectonucleotidase CD39, involved in the adenosine pathway, was upregulated exclusively in class-switched B cells in responders (FIG. 7F). There are data supporting a role for adenosine in facilitating B cell class switching. Therefore, multivariate analysis suggests a relationship between vaccine response and an alternative lineage B cell phenotype with metabolic machinery to facilitate anabolic metabolism fueled by FAO and enhanced class switching stimulated by adenosine.

### Example 4-CD11c+B Cells Dominate Response to COVID Vaccination in SOTRs

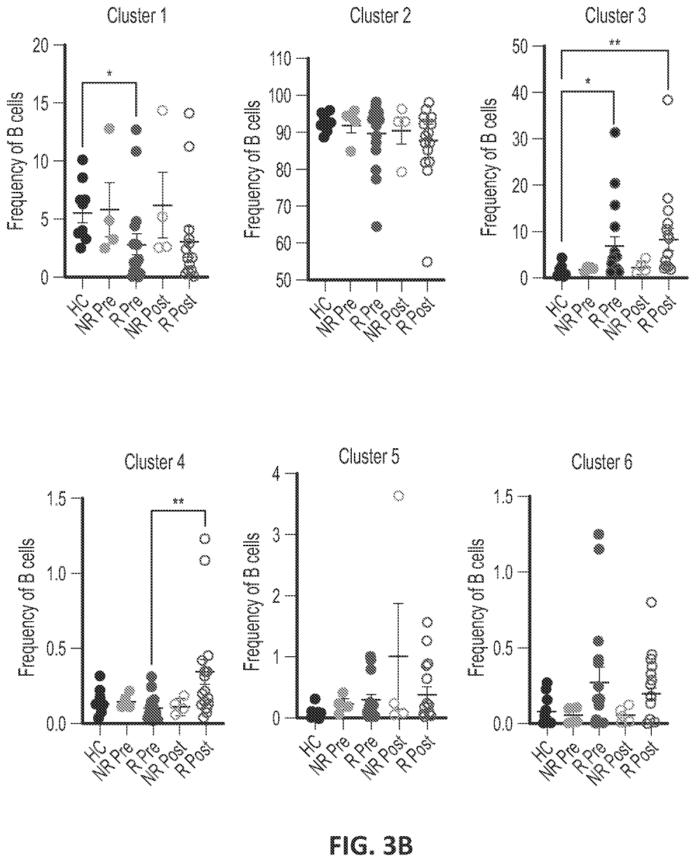

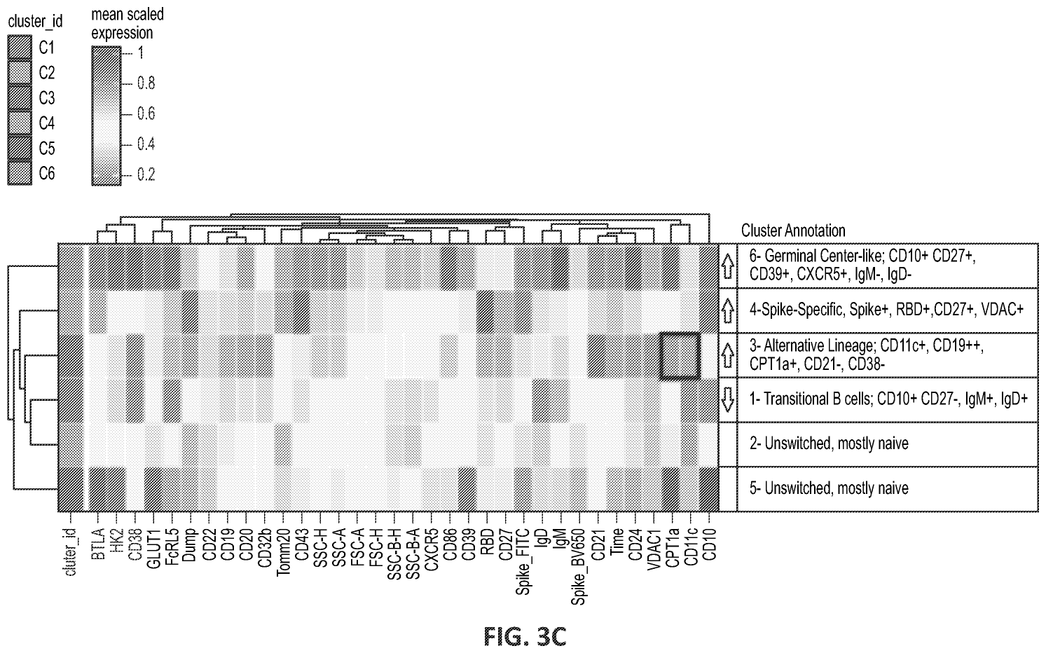

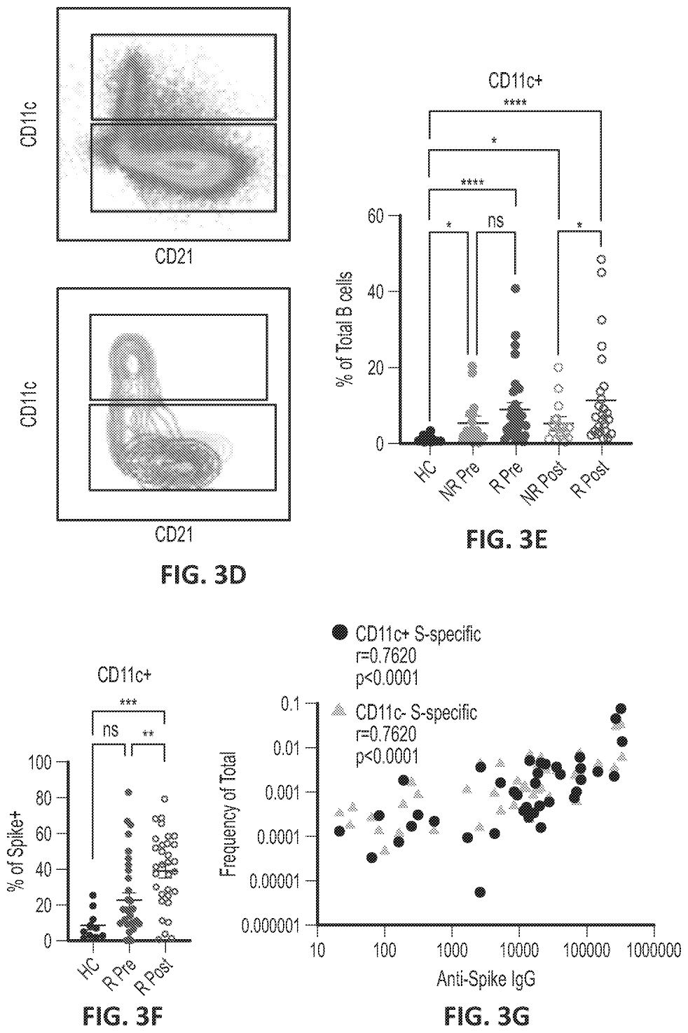

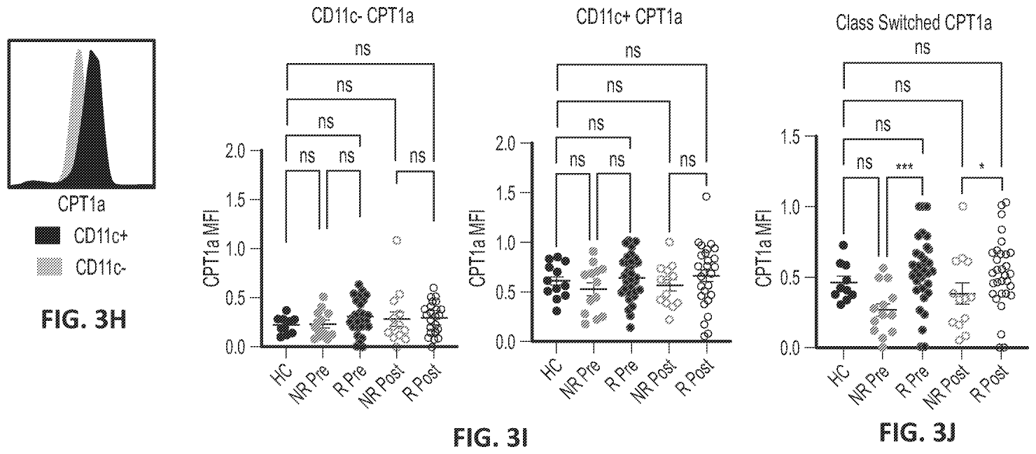

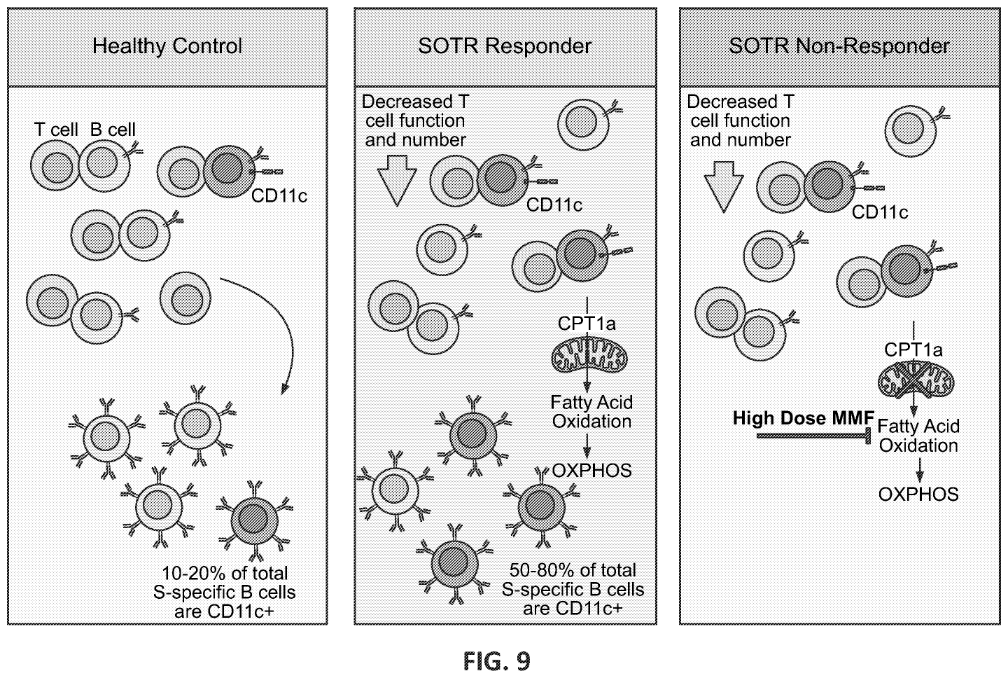

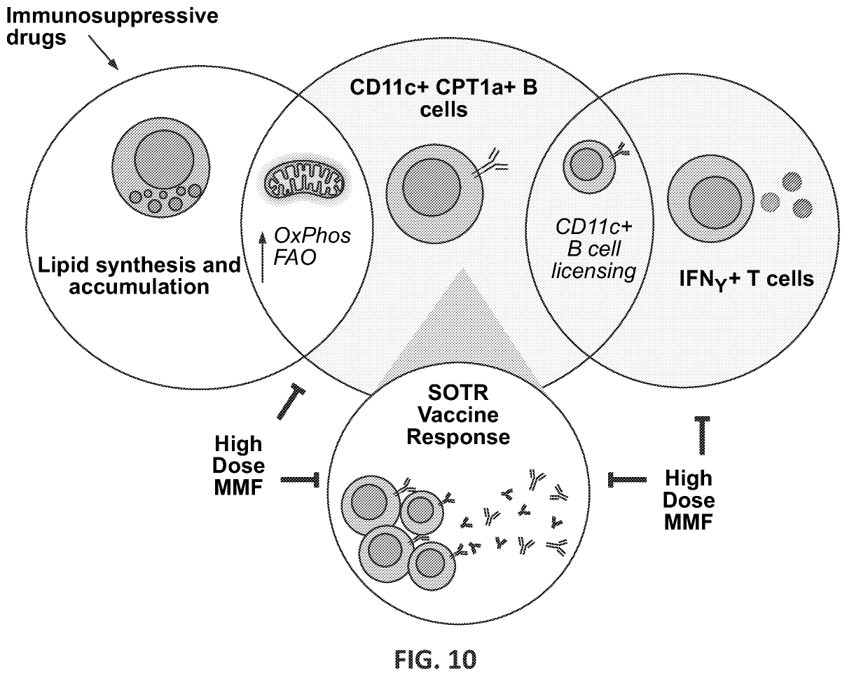

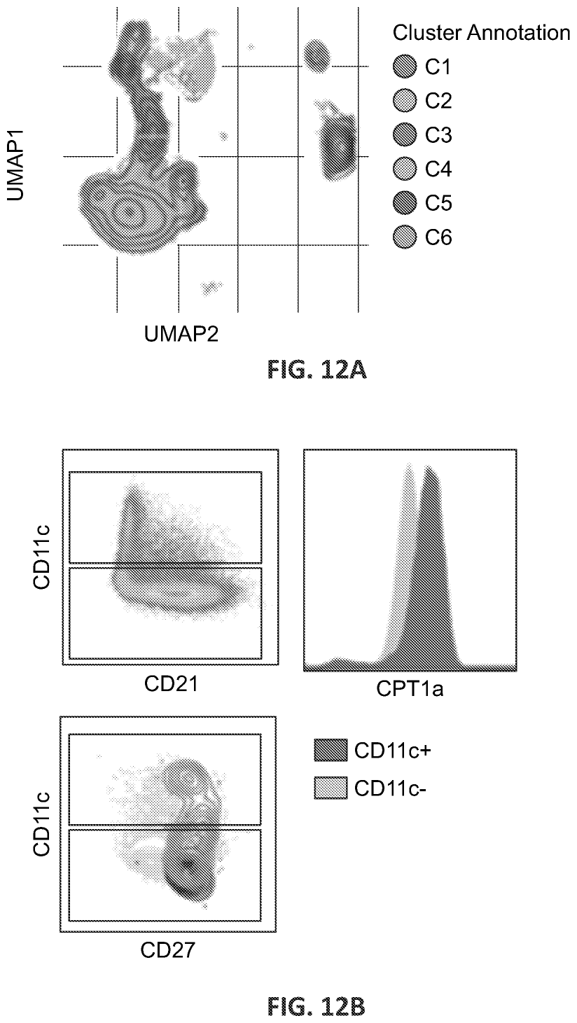

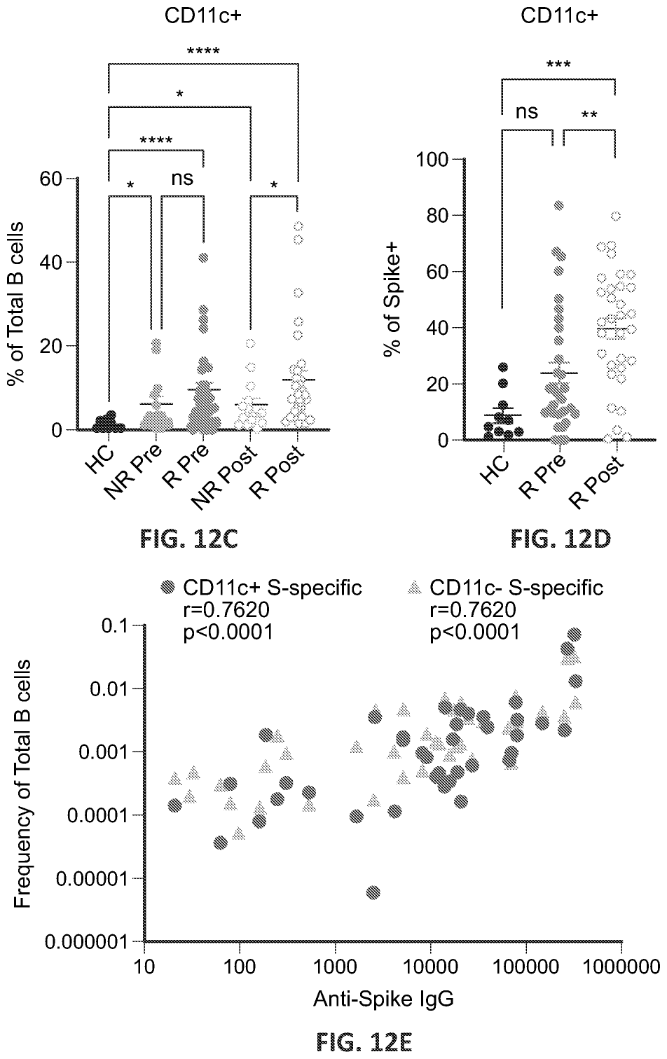

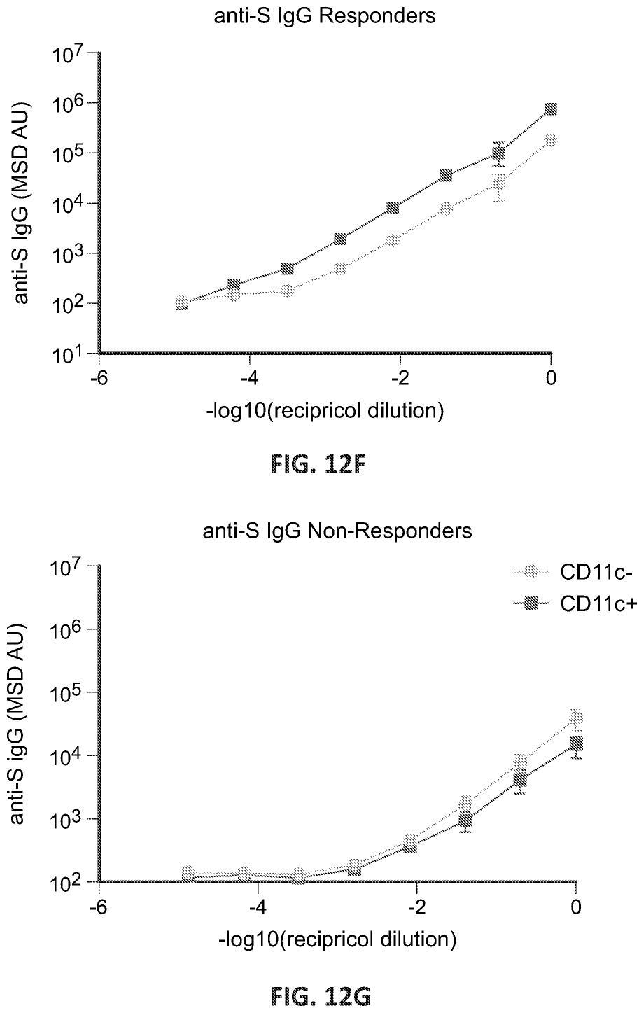

To elucidate how the expression of these proteins relate to each other, both immunological and metabolic phenotypes were evaluated simultaneously using the data reduction methods Uniform Manifold Approximation and Projection (UMAP) projections with unbiased clustering using the FlowSOM algorithm. FlowSOM clustering identified 3 of 6 total clusters of B cells that were differentially expressed (FIGS. 3A-3C). Cluster 4, which was unique to responders post-dose 3, contained S-specific B cells (FIGS. 3A-3C). Cluster 3, defined by co expression of CD11c and CPT1a, was present in responders prior to dose 3 and was expanded two weeks after dose 3. Given the distinct nature of cluster 3, a standard gating scheme was devised to evaluate this population in relation to the S-specific B cells (FIG. 3D). CD11c+B cells were expanded exclusively in responders and minimally present in HCs (FIG. 3E), a pattern maintained in S-specific B cells (FIG. 3F). While up to 80% of the S-specific B cells in SOTRs were CD11c+, only 10-20% of S-specific B cells were CD11c+ in HCs (FIG. 3F). Importantly, both CD11c+S-specific B cells and CD11c-S-specific B cell frequencies correlated with anti-IgG titers in SOTRs (FIG. 3G), demonstrating that both subsets contribute to plasma IgG titers. Therefore, the expansion of alternative lineage CD11c+B cells appears to be a beneficial compensatory mechanism in SOTRs to increase the frequency of S-specific B cells and anti-S titers, rather than a defect responsible for the decreased titers seen in SOTRs compared to HCs. CD11c+B cells have increased T cell stimulatory capacity and more sustained interactions with T cells in the lymph node. Consequently, in the context of immunosuppression whereby activated T cells become a limiting resource, CD11c-B cells may be most able to compete for the limited T cell help available for activation and expansion. Consistent with the cluster 3 phenotype, CD11c+ cells expressed higher CPT1a than CD11c-B cells in both HCs and SOTRs (FIGS. 3H-31). The high expression of CPT1a in class-switched B cells seen via multidimensional analysis was therefore driven largely by expansion of CD11c+B cells, which preferentially express high levels of CPT1a (FIG. 3.J). This finding led to the hypothesis that the immune cell metabolic landscape of SOTRs taking maintenance immunosuppression supported expansion of CD11c+CPT1a+B cells (FIGS. 12A-12G).

### Example 5-High Dose MMF Inhibits Mitochondrial Fatty Acid Oxidation

Given the relationship between MMF treatment and failure to respond, how MMF dose related to B cell immunometabolic phenotype was evaluated, with a particular focus on CD11c and CPT1a. MMF is a prodrug of the anti-metabolite mycophenolic acid (MPA), which inhibits inosine 5′-monophosphate dehydrogenase (IMPDH) to reduce de novo purine synthesis, limiting the availability of guanine and inhibiting lymphocyte proliferation. While this drug has not been extensively investigated for its ability to modulate metabolic programs related to cellular energetics, a reported side effect of MMF is hyperlipidemia, and an intestinal cell model demonstrated that MPA leads to increased intracellular fatty acids and cholesterol. These data suggest MMF may alter lipid metabolism in addition to inhibiting de novo purine synthesis.

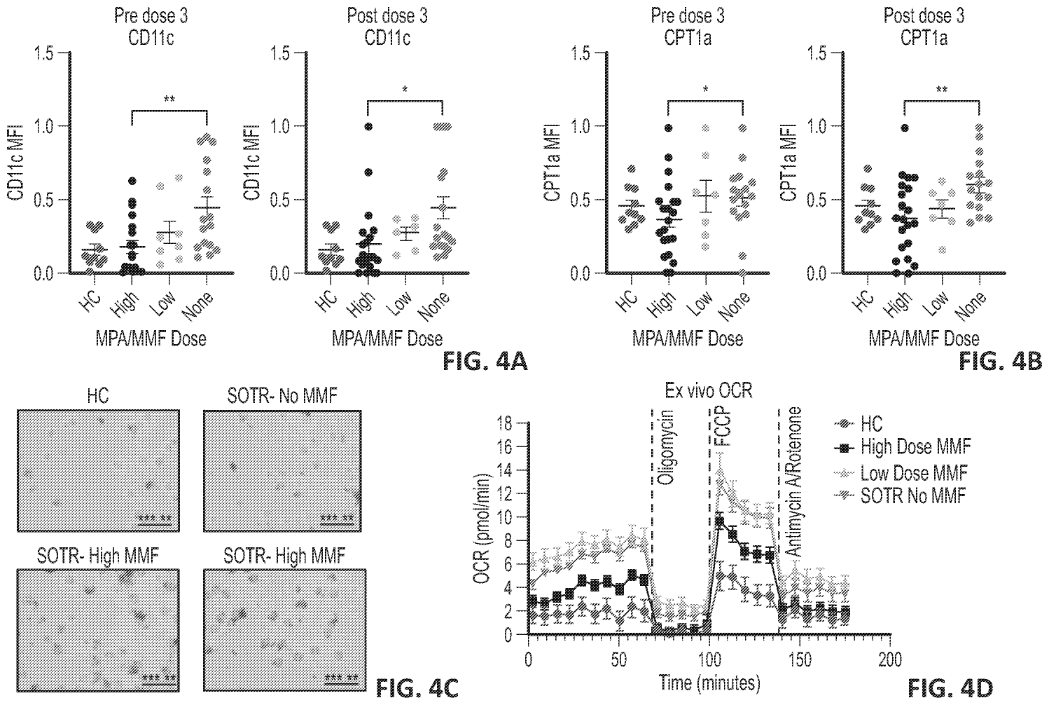

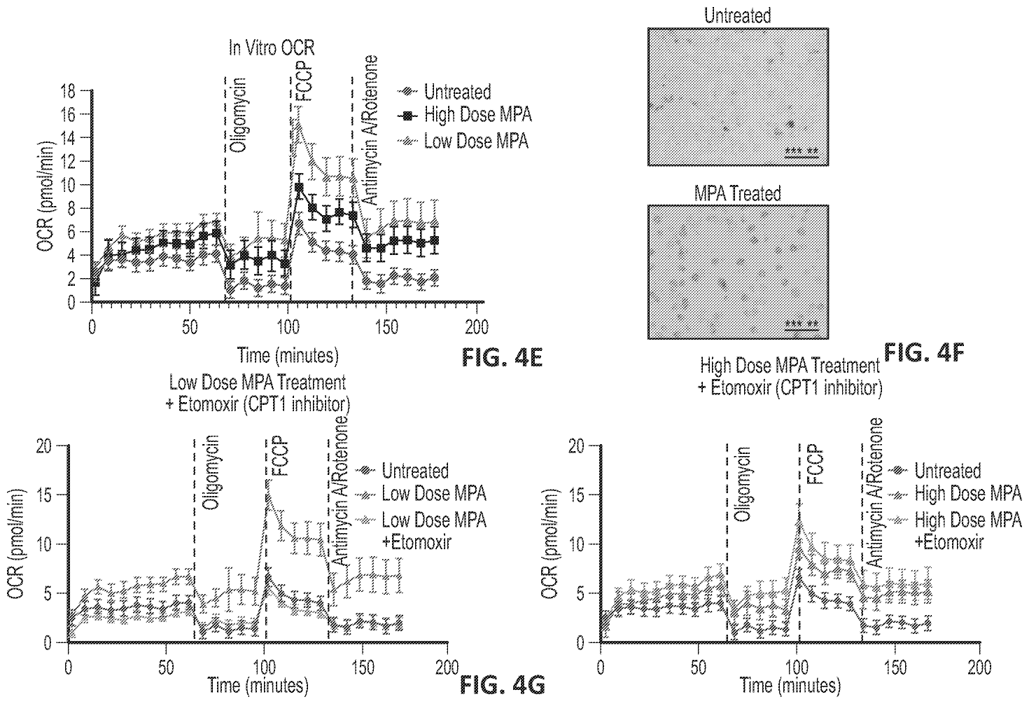

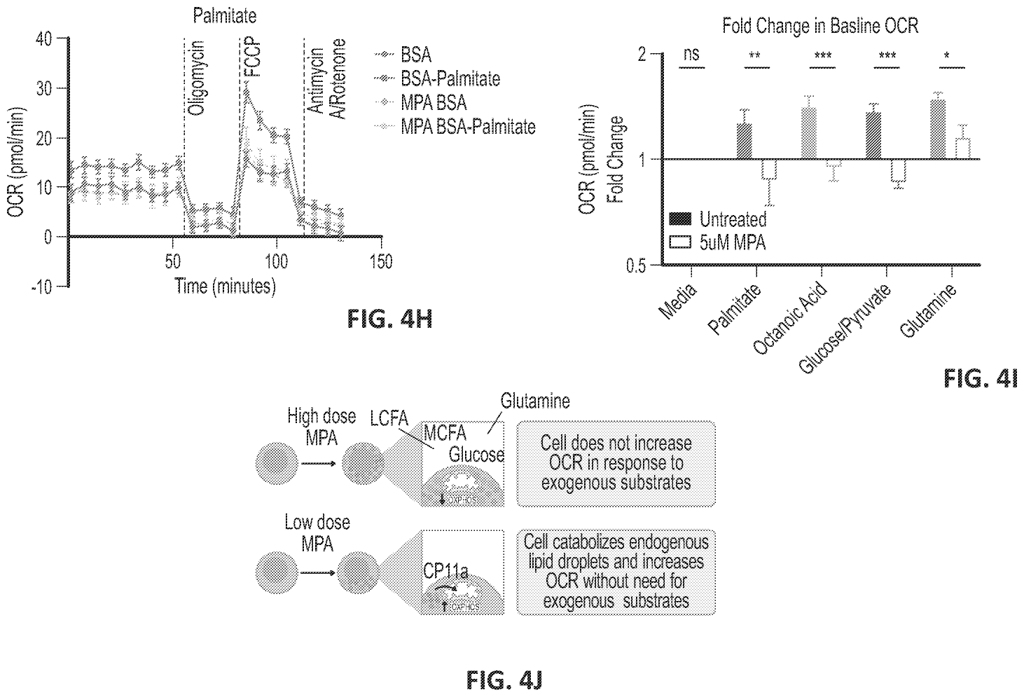

Class switched B cells from SOTRs receiving high dose MMF or MPA (>1000 mg/per day or >721 mg/day, respectively) expressed significantly lower CD11c (FIG. 4A) and CPT1a (FIG. 4B). Of note, the three outliers on high dose MMF with increased CD11c expression were all vaccine responders, suggesting that patients who managed to mount effective vaccine responses despite high dose MMF did so through expansion of CD11c+B cells.

Due to the association with MMF dose and CPT1a expression, PBMCs from SOTRs receiving high dose, low dose, or no MMF were evaluated ex vivo for alterations in lipid metabolism. Immunosuppression dose is largely determined by estimation of graft function and the measurement of the blood levels of immunosuppressive drugs, although neither method is sensitive or specific for determining the current immunosuppressive status, particularly for MMF, where drug levels are not routinely measured. Consistent with a defect in FAO, PBMCs from SOTRs on high dose MMF demonstrated an accumulation of intracellular lipid droplets (FIG. 4C). Mitochondrial bioenergetics were next measured directly ex vivo via the Agilent Seahorse XF Cell Mito Stress Test at basal levels and in response to mitochondrial stress, in the absence of additional stimulation or exogenous substrates. The oxygen consumption rate (OCR) was used as a measurement of mitochondrial respiration/oxidative phosphorylation (OXPHOS). Unexpectedly, PBMCs from SOTRs receiving either low dose MMF or other immunosuppression demonstrated significantly higher basal respiration compared to HCs, without the addition of exogenous substrates (FIG. 4D). In contrast, PBMC of participants on high dose MMF exhibited a reduced OCR compared to other SOTRs, indicating that this enhanced respiration is suppressed in response to high dose MMF.

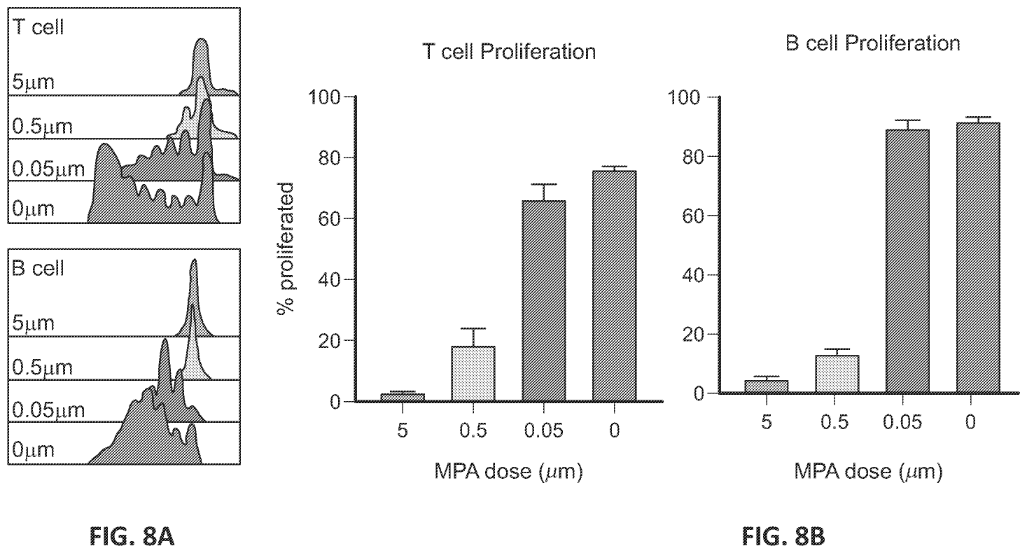

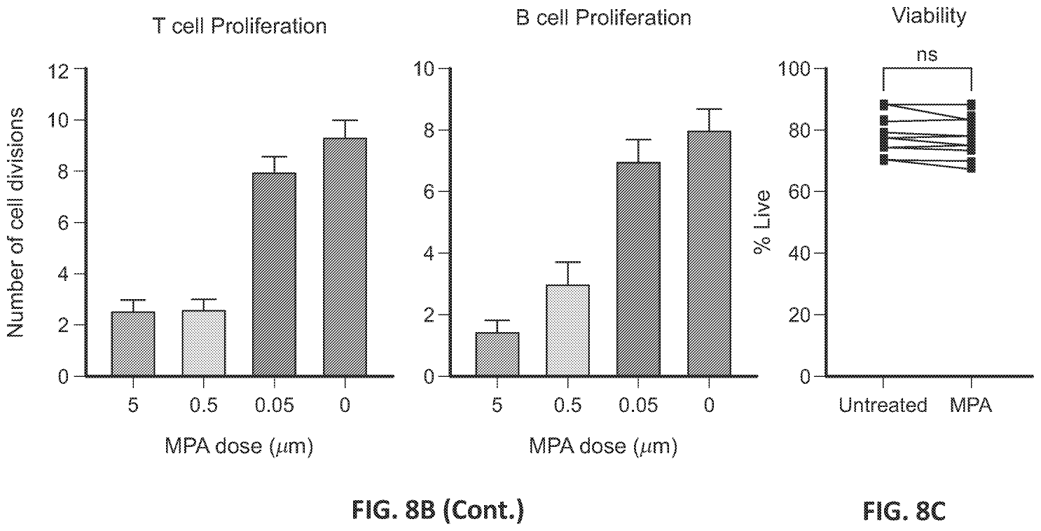

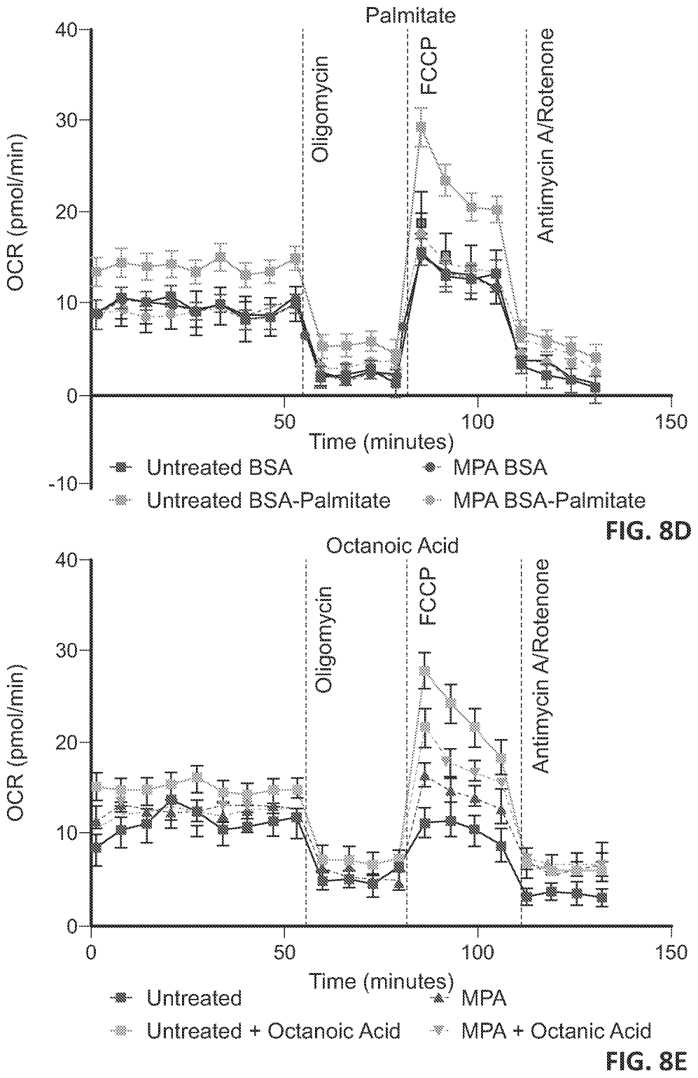

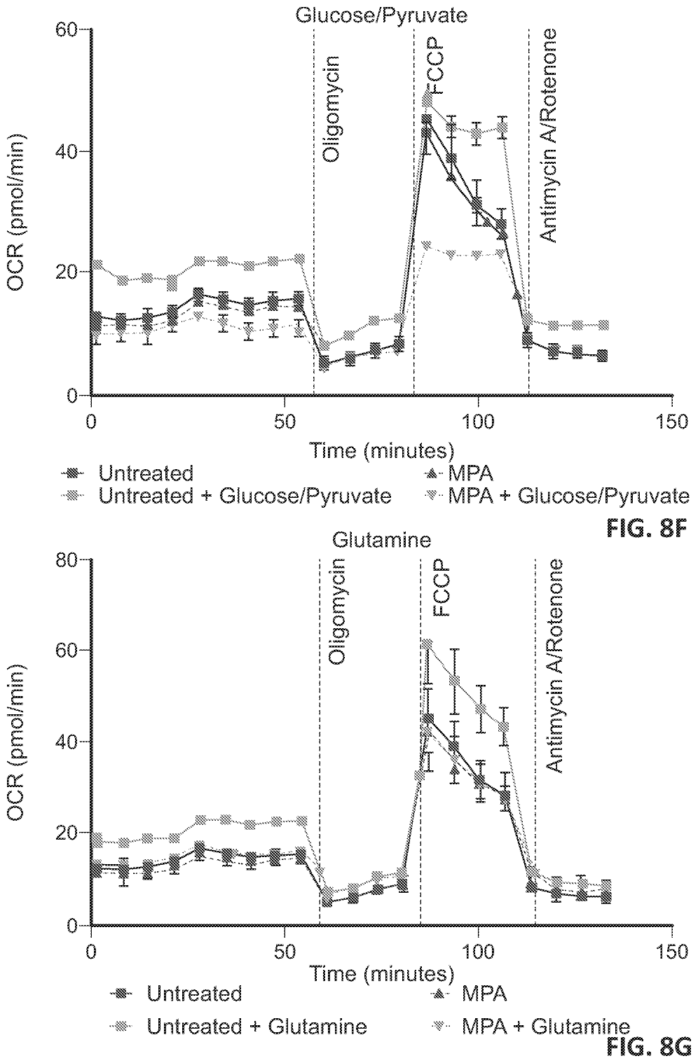

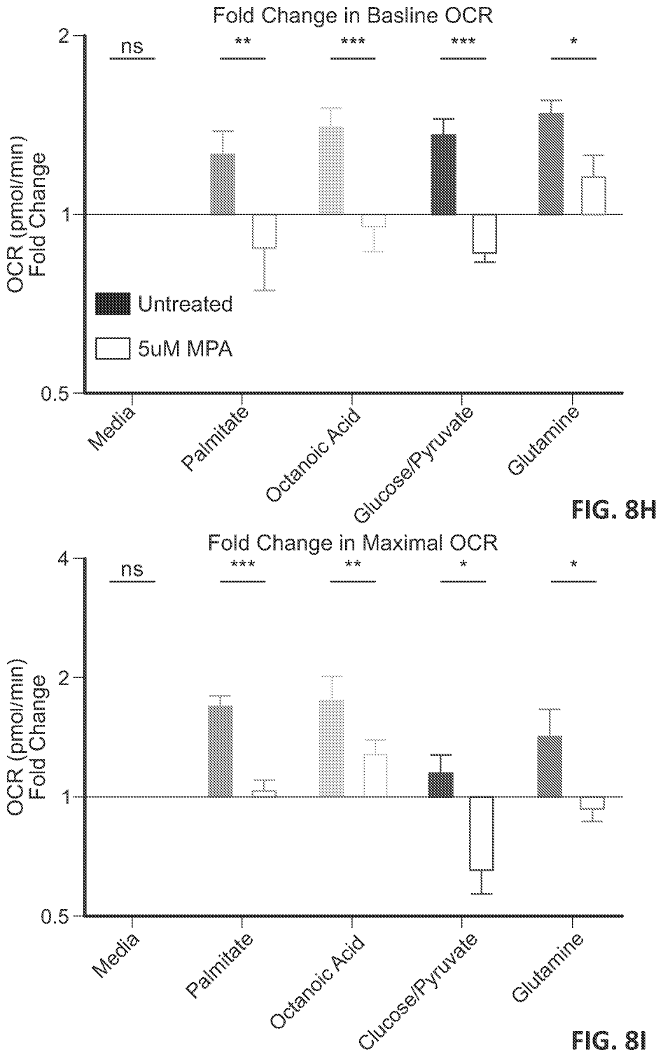

To understand if this metabolic alteration was a direct effect of MMF or due to systemic activation following transplantation, healthy PBMCs were treated in vitro with two doses of MPA to model a range of therapeutic doses; high dose (5 μM) to completely inhibit proliferation and low dose (0.05 μM) to limit proliferation (FIGS. 8A-8B). Low dose MPA increased OCR compared to untreated PBMCs, reproducing the increased OCR seen in PBMCs from SOTRs receiving low dose MMF or other immunosuppressive agents (FIG. 4F). These data indicate the increased OCR ex vivo was driven by administration of MMF and not systemic activation due to transplantation. High dose MPA, on the other hand, demonstrated reduced OCR compared to low dose, consistent with ex vivo data (FIG. 4E). PBMCs treated in vitro with high dose MPA also demonstrated increased accumulation of lipids (FIG. 4F), suggesting that in these cells FA were being stored as triacylglycerols rather than being used to fuel OXPHOS.

The data indicate that cells exposed to high dose MMF in vivo or in vitro fail to oxidize endogenous lipids, and it was therefore postulated that cells treated with low dose MMF were oxidizing endogenous lipid droplets to fuel the increased OCR without the addition of exogenous substrates. To test this hypothesis, cells were exposed to low dose etomoxir, an inhibitor of CPT1a transport of FAs into mitochondria. Etomoxir treatment completely reversed the increase of OCR in low dose MPA-treated cells (FIG. 4G). To confirm that high dose MPA inhibits FAO, palmitate (a long chain FA) was added exogenously to cultures 20 minutes prior to evaluation of mitochondrial respiration. While untreated PBMCs increased OCR in response to palmitate, high dose MPA treated cells were unable to oxidize palmitate (FIG. 4H). Cells treated with high dose MPA responded to mitochondrial stress tests in a comparable manner to untreated cells (FIGS. 4D-4H) and had equivalent cell viability (FIG. 8C), indicating that the cells maintained viable mitochondria. However, high dose MPA resulted in diminished mitochondrial oxidation of various supplemented substrates, including medium-chain fatty acids, glucose, and glutamine (FIGS. 41, 8D-81). Collectively, these data indicate that high dose MMF results in accumulation of intracellular lipid droplets due to failure of robust mitochondrial lipid oxidation, while low dose MMF results in clearance of intracellular lipid droplets due to enhanced mitochondrial lipid oxidation. Therefore, these data suggest careful consideration should be placed on the amount of MMF an individual truly requires to prevent transplant rejection, and for those who require higher doses, alternative therapies to enhance FAO should be investigated to improve vaccination responses.

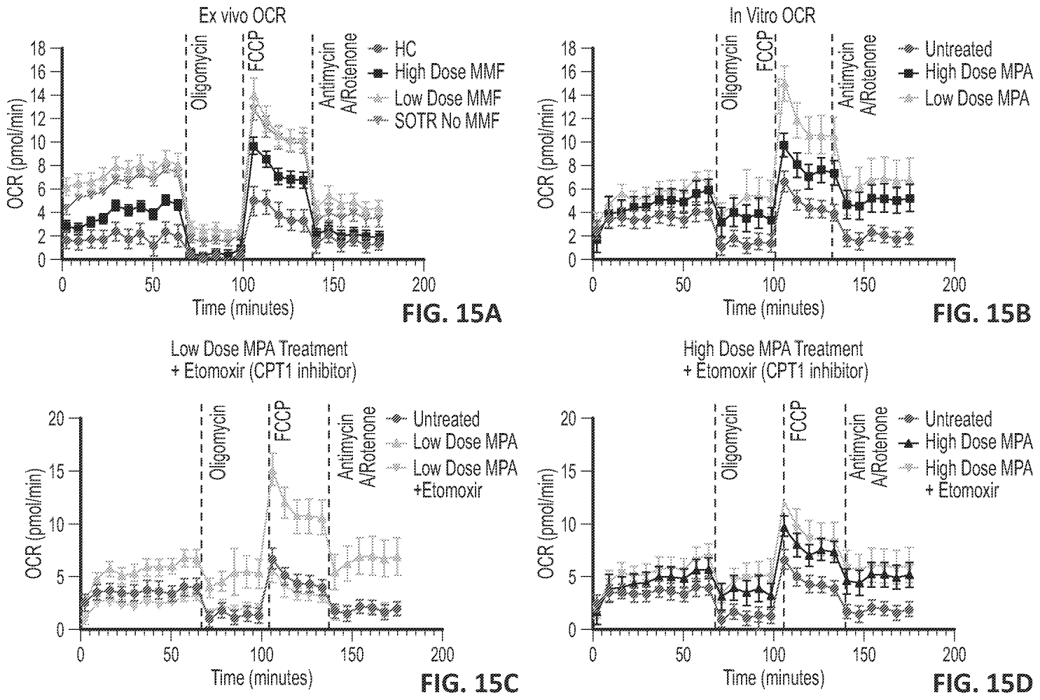

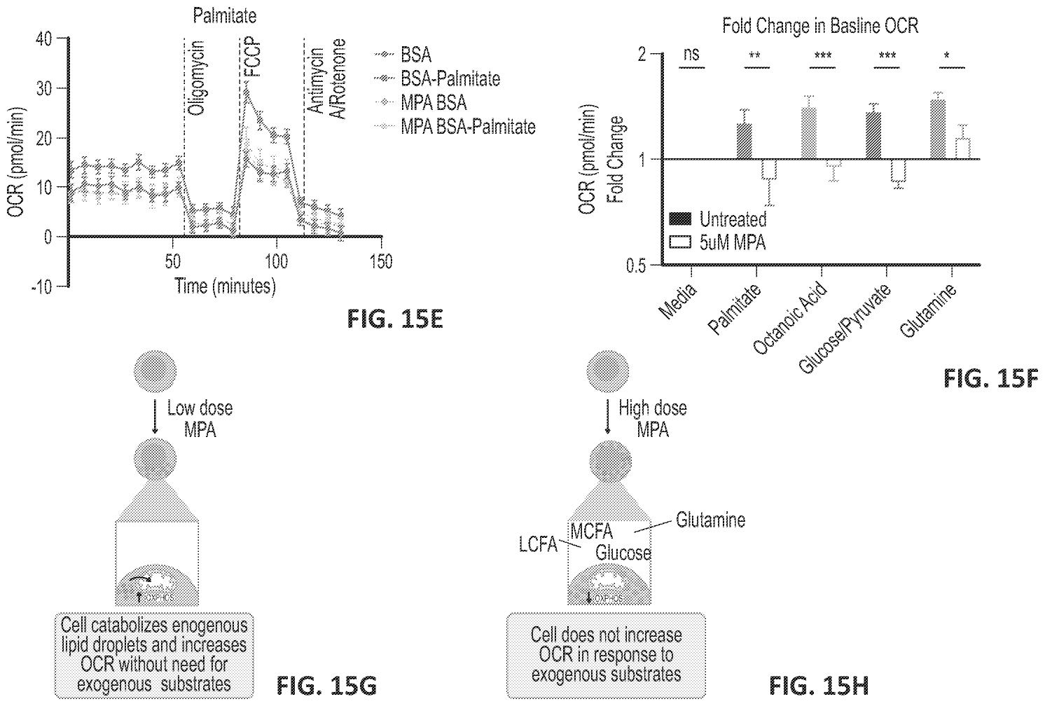

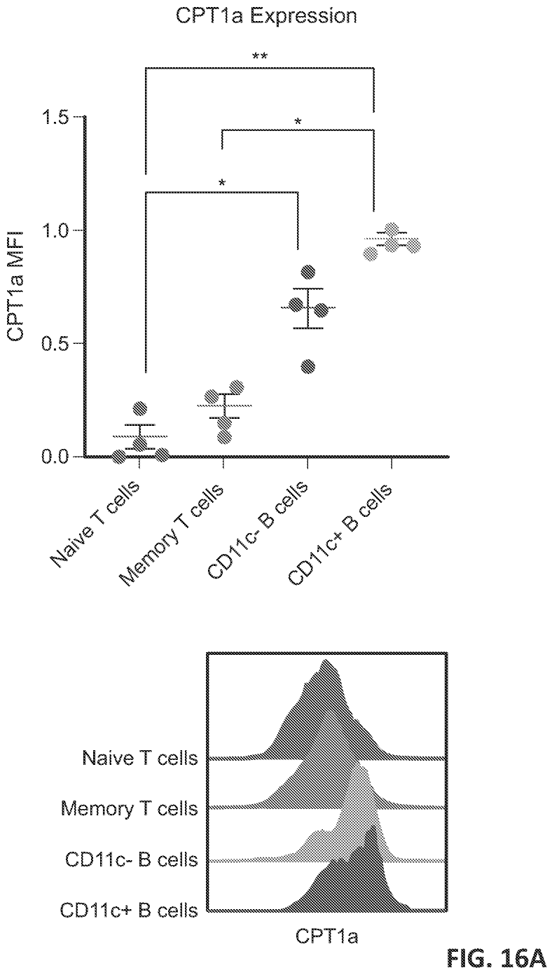

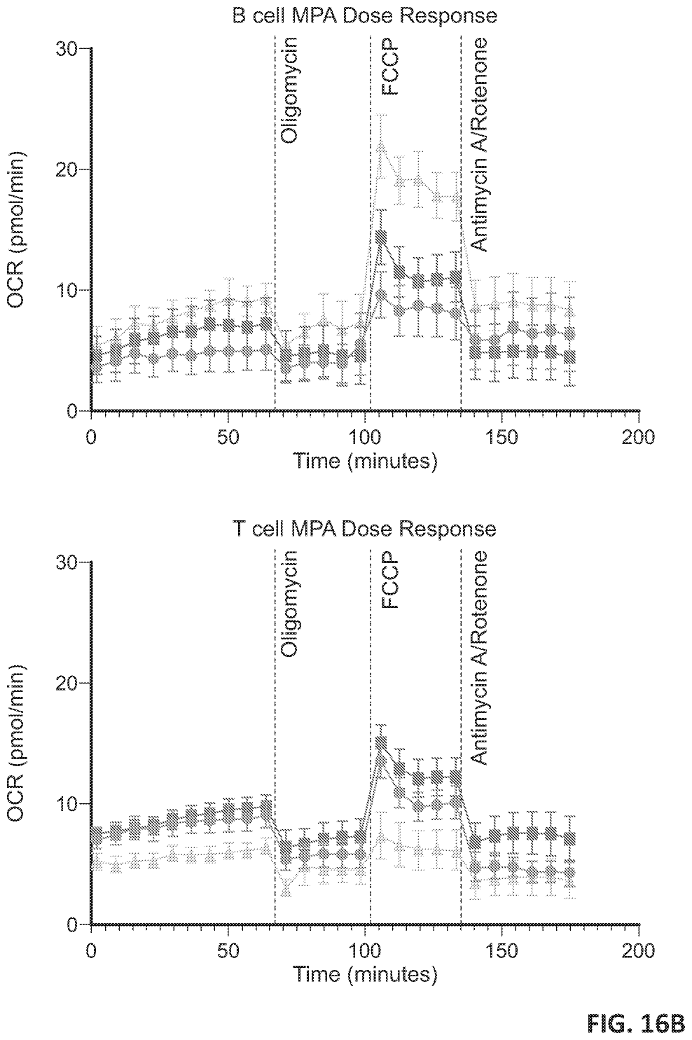

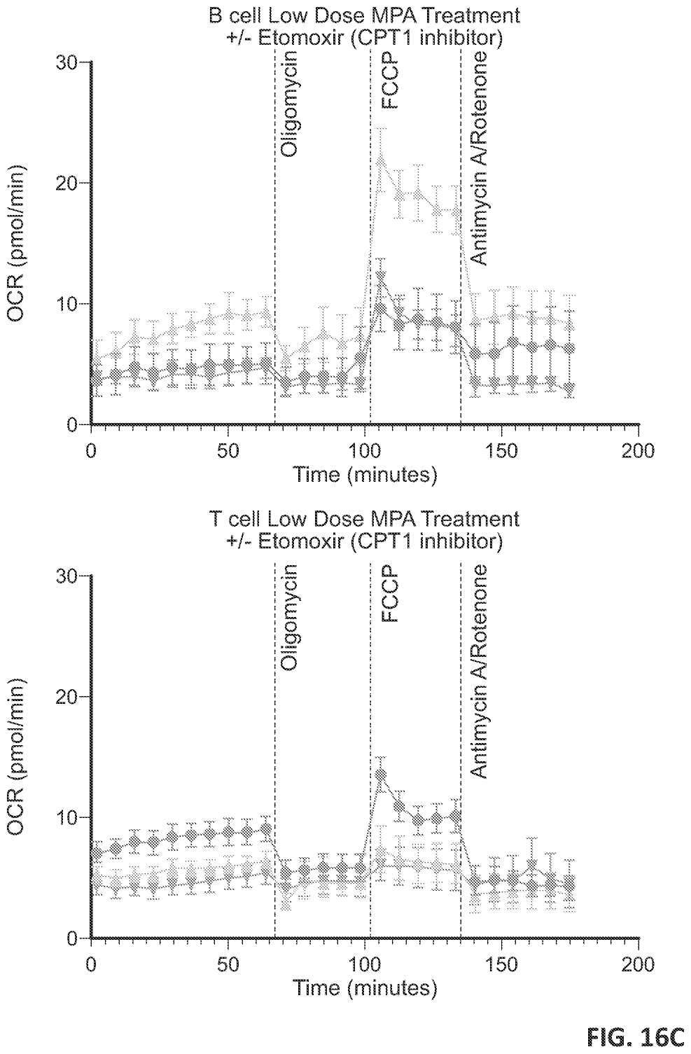

Here, the underlying mechanisms of response to COVID-19 vaccination in SOTRs were explored by evaluating the immunologic and metabolic phenotypes of B cells. Using this approach, an expanded population of alternative lineage S-specific CD11c-B cells was identified in SOTRs that utilizes FAO. These B cells were not expanded in HCs and therefore likely represent a distinct vaccine response that is not required in the absence of immunosuppression. Furthermore, immunosuppression increased FAO-dependent mitochondrial metabolism, which supports alternative lineage B cells with high CPT1a expression (FIGS. 16A-16C). Finally, MMF appears to induce a hormetic effect, whereby low doses induced increased FAO and high doses inhibited FAO and mitochondrial response to exogenous substrates (FIGS. 4J, 9, 10). These data suggest that dose reduction of MMF prior to vaccination or use of nutritional/pharmaceutical enhancement of FAO should be investigated as methods to improve vaccine responses in immunocompromised patient populations (FIGS. 15A-15H).

### Example 6-scRNA Seg Reveals Increased Oxidative Phosphorylation and Fatty Acid Oxidation in CD11c+B Cells from SOTRs

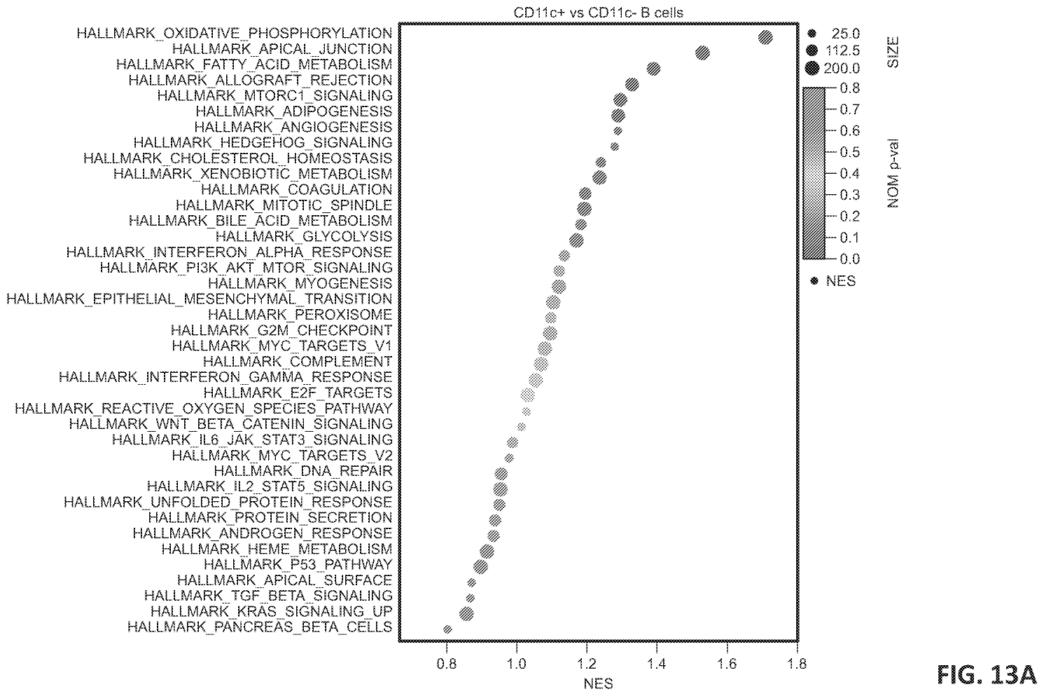

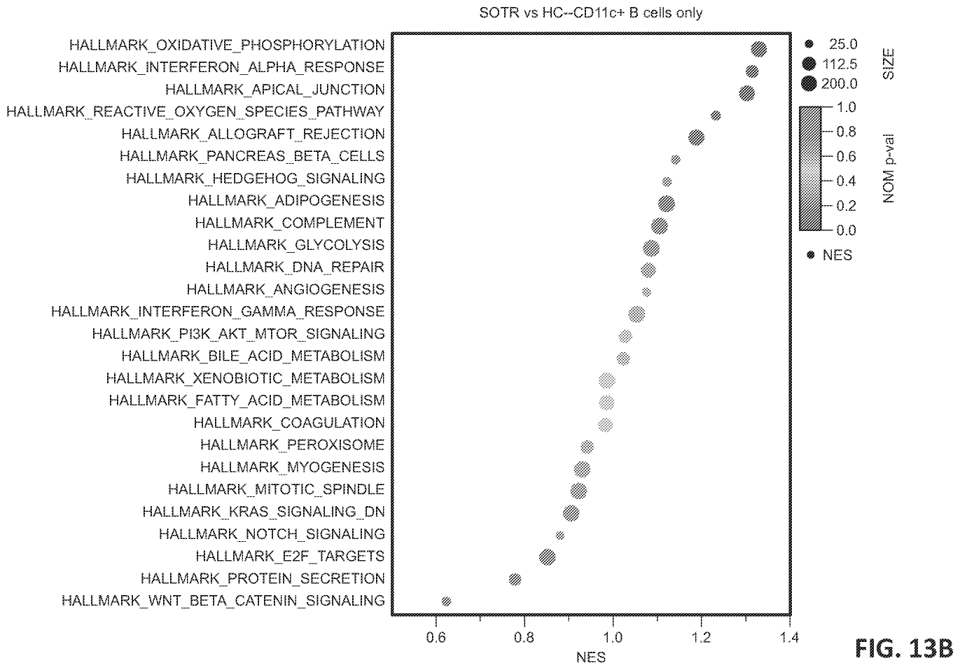

Gene set enrichment was performed on total B cells comparing CD11c+B cells to CD11c-B cells from SOTRs and HCs (FIG. 13A). Gene set enrichment was also performed on CD11c+B cells comparing SOTR to HCs (FIG. 13B).

### Example 7-Immunosuppression Induces Lipid Synthesis and Accumulation

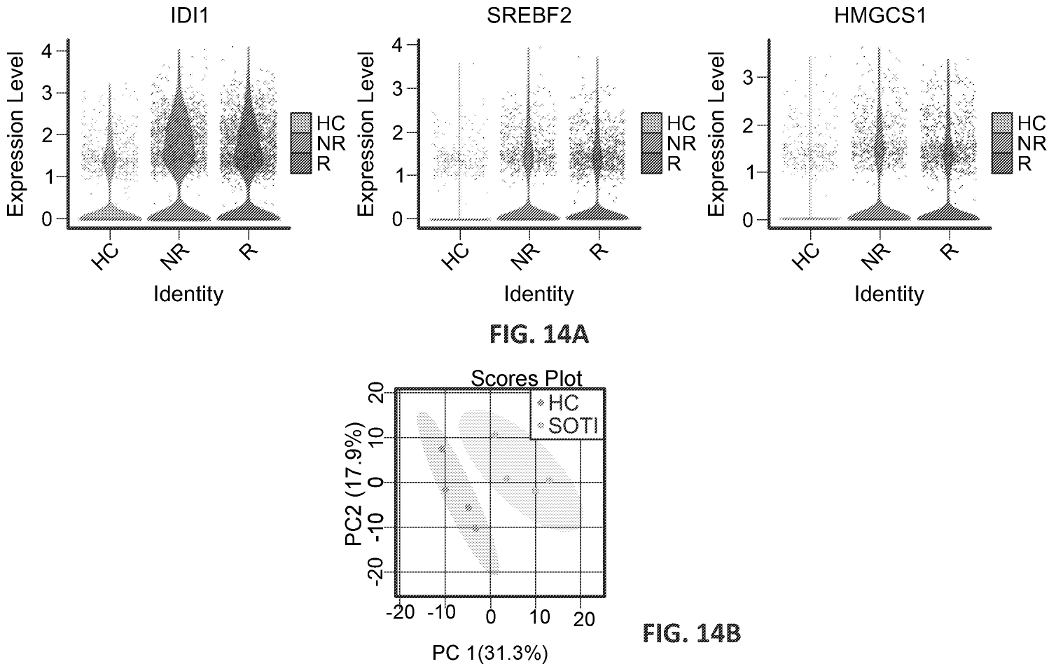

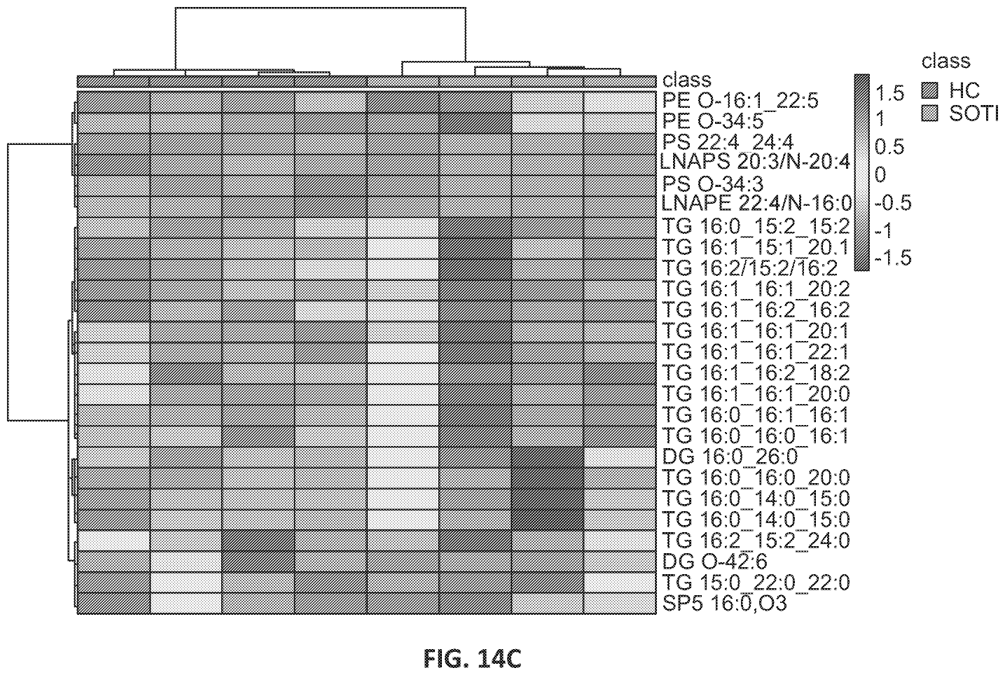

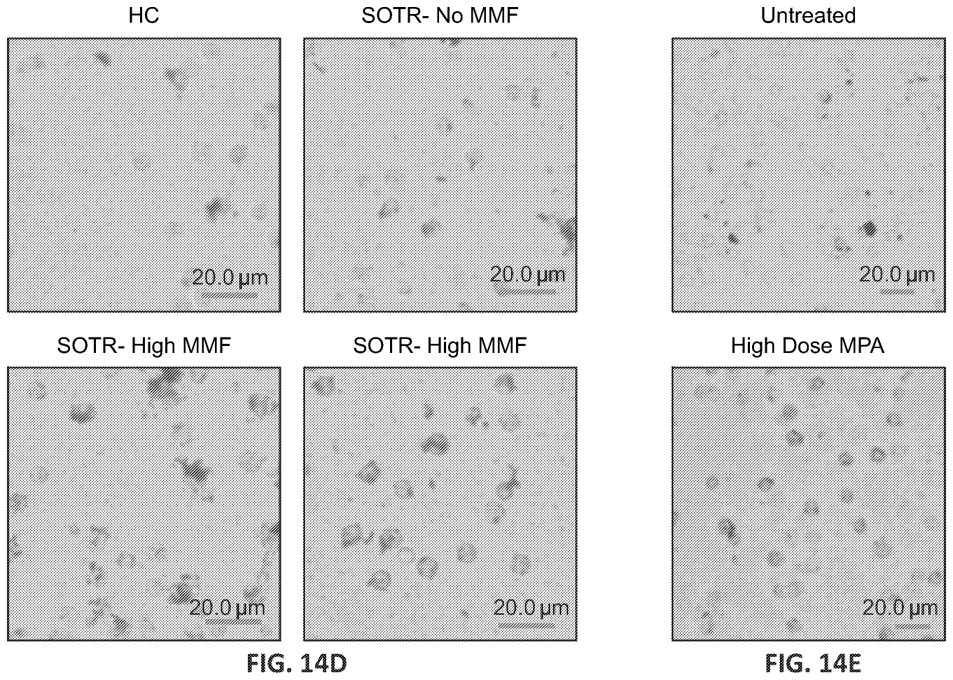

Genes associated lipid synthesis were increased in total B cells from SOTRs (R or NR) in SCR.NA seq (FIG. 14A). PCA plot of lipidomics performed on total B cells derived from SOTRs or HCs (FIG. 14B). Hierarchical clustering of top 20 differentially expressed lipids in B cells from SOTRs and HCs demonstrate clear segregation in lipid profiles (FIG. 14C). Accumulation of lipid droplets were measured by Oil Red O in PBMCs from HC or SOTR (FIG. 14D). Results show that there were higher levels of Oil Red O staining in individuals on high dose MMF. Accumulation of lipid droplets was seen by Oil Red O staining in healthy cells treated with high dose of mycophenolic acid (active ingredient in MMF) (FIG. 14E).

### Example 8-IFNg Supports CD11c+B Cell Differentiation and Response to Vaccination

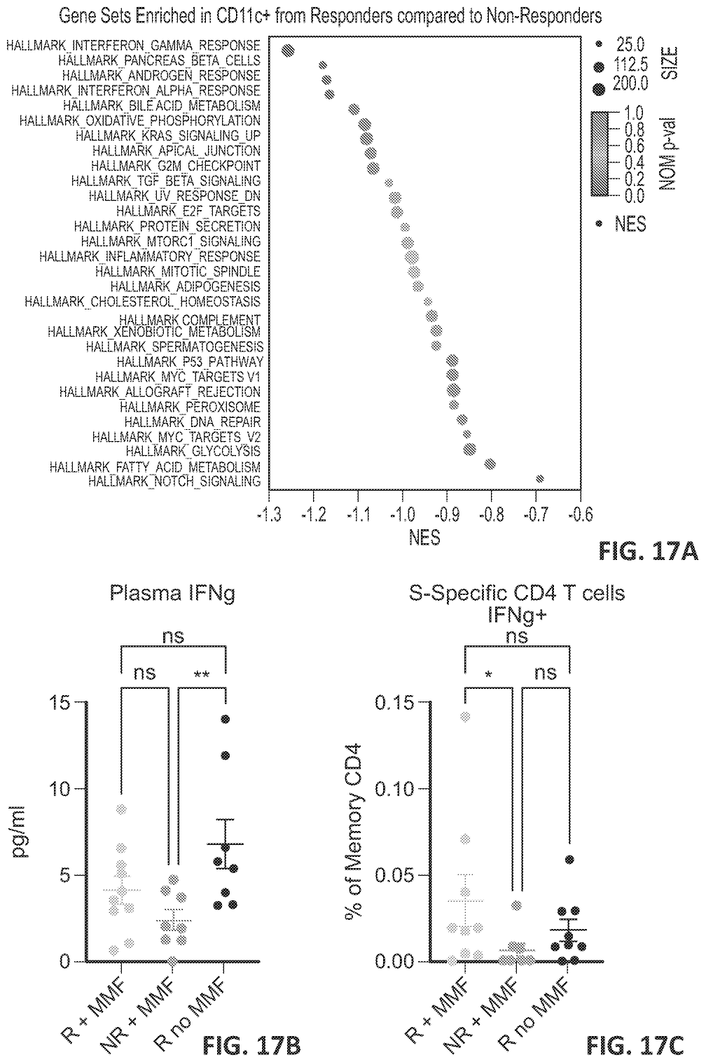

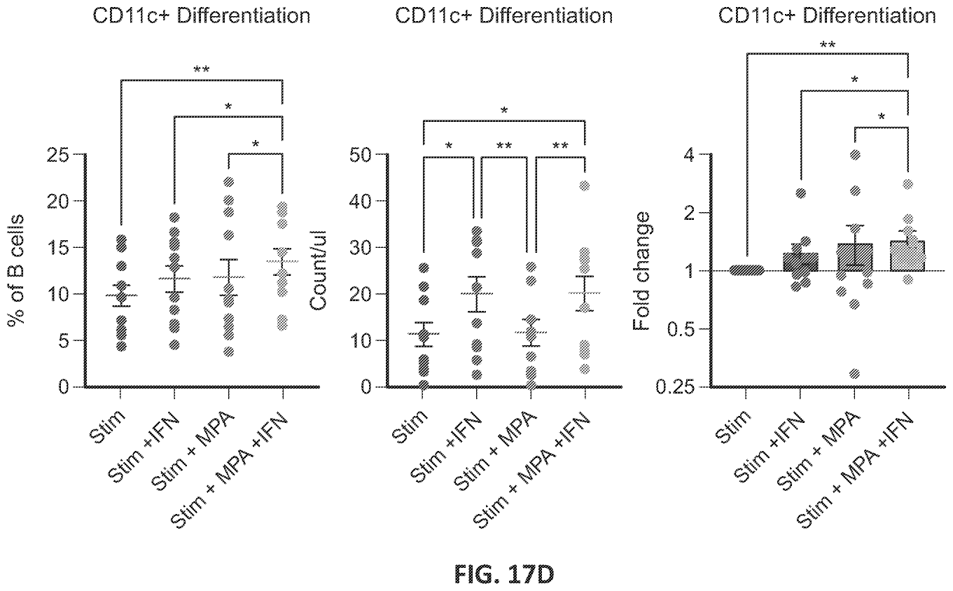

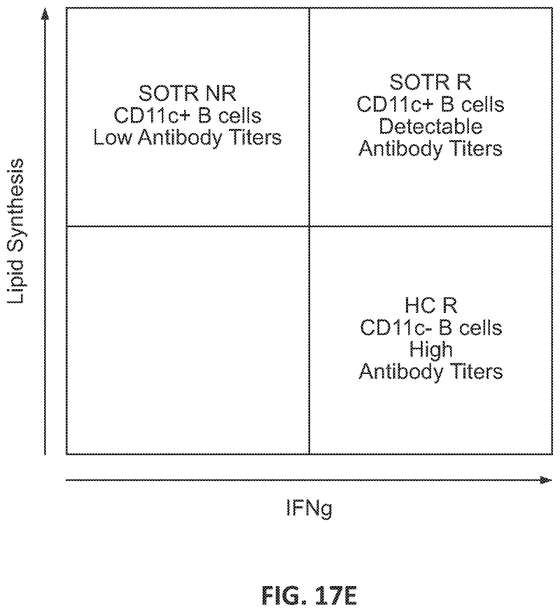

Gene set enrichment on CD11c+B cells from SOTR responders compared to non-responders demonstrates increased IFNg signaling in responders (FIG. 17A), wherein plasma IFNg levels were increased in responders (FIG. 17B) and S-specific T cells that produce IFNg were increased in responders (FIG. 17C). Healthy B cells were provided polyclonal stimulation (CpG, IL-21, CD40L) for four days in the presence or absence of low dose MPA and IFNg (FIG. 17D). The frequency of CD11c+B cells in culture was evaluated at day 4, wherein IFNg and MPA combined induced the highest levels of CD11c+B cells. Results proposed dichotomy of lipid synthesis driving CD11c expression on B cells and requirement for IFNg to generate detectable antibody responses in SOTRs (FIG. 17E).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of measuring effects of immune suppression in a subject, the method comprising:
(a) quantifying a number of immune cells in a biological sample of the subject;
(b) determining an expression level of a protein from the biological sample; and
(c) analyzing the number of immune cells and the expression level of the protein, thereby measuring the effects of immune suppression in the subject.

2. The method of claim 1, wherein the subject is immunocompromised.

3. The method of claim 1, wherein the subject is a solid organ transplant recipient (SOTR).

4. The method of claim 1, wherein the subject has an autoimmune disease.

5. The method of claim 1, wherein the subject is administered an immunosuppressive agent.

6. The method of claim 5, wherein the immunosuppressive agent is mycophenolate motefil (MMF).

7. The method of claim 1, wherein the immune cell is a B cell.

8. The method of claim 7, wherein the B cell is a CD11c+B cell.

9. The method of claim 1, wherein the protein is an immunometabolic marker on a B cell.

10. The method of claim 9, wherein the immunometabolic marker is selected from the group consisting of CD19, CD20, CD10, CD27, CD21, IgM, IgD, CD24, CD38, CD43, CD86, CXCR5, CD11c, CD39, FcRL5, BTLA, CD22, CD32, CD3, CD14, CPT1a, Hexokinase II, VDAC1, Tomm20, GLUT1, or any combinations thereof.

11. The method of claim 9, wherein the immunometabolic marker is selected from CPT1a, HK2, CD11c, FcRL5, CD39, or any combinations thereof.

12. The method of claim 9, wherein the immunometabolic marker is CPT1a.

13. The method of claim 1, wherein the analyzing step (c) comprises identifying the number of immune cells and the expression level of the protein, and using the number of immune cells and the expression level of the protein in combination to determine an immune response in the subject.

14. The method of claim 13, wherein the immune response comprises determining the subject's ability to respond to a vaccination or infection.

15. The method of claim 14, wherein the vaccination is a COVID-19 vaccination.

16. The method of claim 1, wherein the analyzing step (c) comprises performing flow cytometry.

17. The method of claim 1, wherein the biological sample is blood plasma.
